(19) United States
(12) Patent Application Publication
(10) Pub. No.: US 2018/0207293 A1
(43) Pub. Date: Jul. 26, 2018

(54) NANOPARTICLES FOR SUSTAINED OPHTHALMIC DRUG DELIVERY AND METHODS OF USE

(71) Applicant: 2C TECH CORP., Irvine, CA (US)

(72) Inventors: Robert W. SHIMIZU, San Juan Capistrano, CA (US); Elizabeth Woldemussie, Laguna Niguel, CA (US); James P. Currie, Trabuco Canyon, CA (US); Richard Kanner, Irvine, CA (US)

(21) Appl. No.: 15/604,361

(22) Filed: May 24, 2017

Related U.S. Application Data
(60) Provisional application No. 62/450,265, filed on Jan. 25, 2017.
(60) Provisional application No. 62/459,439, filed on Feb. 15, 2017.

(51) Int. Cl. A61K 47/69; A61K 9/00; C07K 5/083; C07K 5/062; A61K 38/48; A61K 38/46; A61K 38/44; A61K 38/39; A61K 38/18; A61K 38/17; A61K 31/7048; A61K 31/575; A61K 31/573; A61K 31/5575; A61K 31/55; A61K 31/542; A61K 31/522; A61K 31/496; A61K 31/4709; A61K 31/4704; A61K 31/382; A61K 31/277; A61K 31/122; A61K 9/51

(52) U.S. Cl. CPC A61K 47/6929; C12Y 301/01064; A61K 9/0048; C12Y 106/99003; C07K 5/081; C07K 5/06026; A61K 38/484; A61K 38/465; A61K 38/44; A61K 38/39; A61K 38/1891; A61K 38/1866; A61K 38/185; A61K 38/18; A61K 38/177; A61K 38/1709; A61K 31/7048; A61K 31/575; A61K 31/573; A61K 31/5575; A61K 31/55; A61K 31/542; A61K 31/522; A61K 31/496; A61K 31/4709; A61K 31/4704; A61K 31/382; A61K 31/277; A61K 31/122; A61K 9/5115; C12Y 304/21007

(57) ABSTRACT

Disclosed is a method of treating an ocular disorder, comprising contacting the eye of a subject in need thereof with an effective amount of a therapeutic nanoparticle composition, the therapeutic nanoparticle composition comprising (i) at least one population of nanostructures, (ii) a peptide attached to the at least at least one population of nanostructures, (iii) a therapeutic agent useful for the treatment of the ocular disorder attached to the at least one population of nanostructures or to the peptide; and (iv) optionally, a linkage between the at least one population of nanostructures or the peptide and the therapeutic agent.

### BACKGROUND OF THE INVENTION

### Field of the Invention

The invention is in the field of ophthalmology. Disclosed is a method of treating an ocular disorder, comprising contacting the eye of a subject in need thereof with an effective amount of a therapeutic nanoparticle composition, the therapeutic nanoparticle composition comprising (i) at least one population of nanostructures, (ii) a peptide attached to the at least at least one population of nanostructures, (iii) a therapeutic agent useful for the treatment of the ocular disorder attached to the at least one population of nanostructures or to the peptide, and (iv) optionally, a linkage between the at least one population of nanostructures or the peptide and the therapeutic agent.

### Background Art

U.S. Pat. No. 6,685,730 discloses methods for the localized delivery of heat and the use thereof to repair tissue. The method involves localized induction of hyperthermia in a tissue by delivering nanoparticles to the tissue and exposing the nanoparticles to an excitation source under conditions whereby they emit heat. The generation of heat effects the joining of the tissue.

U.S. Pat. No. 8,535,681 discloses a drug composition comprising a charged moiety coupled to a therapeutic compound. The charged moiety is configured to interact with at least one type of component of opposite charge in a biological tissue to create an in situ depot for prolonged drug delivery. The biological tissue may be eye tissue or any tissue containing charged components. Further, a method of treating the human body is disclosed. The method is for introducing into a human body a drug composition comprising a charged moiety coupled to a therapeutic compound.

U.S. Pat. No. 8,283,179 discloses functionalized fluorescent nanocrystal compositions and methods for making these compositions. The compositions are fluorescent nanocrystals coated with at least one material. The coating material has chemical compounds or ligands with functional groups or moieties with conjugated electrons and moieties for imparting solubility to coated fluorescent nanocrystals in aqueous solutions. The coating material provides for functionalized fluorescent nanocrystal compositions which are water soluble, chemically stable, and emit light with a high quantum yield and/or luminescence efficiency when excited with light. The coating material may also have chemical compounds or ligands with moieties for bonding to target molecules and cells as well as moieties for cross-linking the coating. In the presence of reagents suitable for reacting to form capping layers, the compounds in the coating may form a capping layer on the fluorescent nanocrystal with the coating compounds operably bonded to the capping layer.

### BRIEF SUMMARY OF THE INVENTION

Provided is a method of treating an ocular disorder, comprising contacting the eye of a subject in need thereof with an effective amount of a therapeutic nanoparticle composition, the therapeutic nanoparticle composition comprising (i) at least one population of nanostructures, (ii) a peptide attached to the at least one population of nanostructures, and (iii) a therapeutic agent useful for the treatment of the ocular disorder attached to the at least one population of nanostructures or to the peptide. The invention enhances the therapeutic utility of the drug active by increasing the duration the active is present in the ocular tissue and/or releases drug under conditions present in the tissue during the diseased state. The invention is based in part on the unexpected discovery that the nanoparticle compositions provided long residence in the vitreous of the eye. The long residence in the eye allows for infrequent dosing, for example, once every 1-4 weeks. In another embodiment, the therapeutic nanoparticle composition is administered once every 1, 2, 3, 4, 5, 6, 7, 8, 9, 10 or more weeks.

The therapeutic nanoparticle compositions comprise peptide coatings on their surface that allow for various linkage chemistries tailored to a particular drug and disease state. By controlling the particle size, one may facilitate distribution of the nanoparticle composition to target tissue and subsequent elimination. In addition, the peptide coatings can be tuned to enhance retention at the site of action. Also, the coatings allow for the development of a colloidal solution that, compared to larger particles, reduces the possibility of the nanoparticle composition interfering with eyesight.

In one embodiment, the therapeutic agent is selected from the group consisting of an antibody, a protein, a nucleic acid and a small organic molecule. In another embodiment, the therapeutic agent is selected from the group consisting of an anti-inflammatory, an anti-infective, an anti-viral, a calcium channel blocker, a neuroprotective agent, a growth factor, a growth factor antagonist, an intraocular pressure lowering drug, and an antineoplastic drug.

In one embodiment, the ocular disorder is selected from the group consisting of glaucoma including Open Angle Glaucoma (e.g., Primary Open Angle Glaucoma, Pigmentary Glaucoma, Exfoliative Glaucoma, and Low Tension Glaucoma), Angle Closure Glaucoma (also known clinically as closed angle glaucoma, narrow angle glaucoma, pupillary block glaucoma, and ciliary block glaucoma) (e.g., Acute Angle Closure Glaucoma and Chronic Angle Closure Glaucoma), Aniridic Glaucoma, Congenital Glaucoma, Juvenile Glaucoma, Lens-Induced Glaucoma, Neovascular Glaucoma, Post-Traumatic Glaucoma, Steroid-Induced Glaucoma, Sturge-Weber Syndrome Glaucoma, and Uveitis-Induced Glaucoma, diabetic retinopathy, macular degeneration, choroidal neovascularization, vascular occlusion, vascular leak, retinal edema, bacterial conjunctivitis, fungal conjunctivitis, viral conjunctivitis, allergic conjunctivitis, uveitis, keratic precipitates, macular edema, inflammation response after intra-ocular lens implantation, uveitis syndromes (e.g., chronic iridocyclitis or chronic endophthalmitis), retinal vasculitis (e.g., as seen in rheumatoid arthritis, juvenile rheumatoid arthritis, systemic lupus erythymatosus, progressive systemic sclerosis, polyarteritis nodosa, Wegener's granulomatosis, temporal arteritis, Adamantiades Bechcet disease, Sjorgen's, relapsing polychondritis and HLA-B27 associated spondylitis), sarcoidosis, Eales disease, acute retinal necrosis, Vogt Koyanaki Harada syndrome, ocular toxoplasmosis, radiation retinopathy, proliferative vitreoretinopathy, endophthalmitis, ocular glaucomas (e.g., inflammatory glaucomas), optic neuritis, ischemic optic neuropathy, thyroid associated orbitopathy, orbital pseudotumor, pigment dispersion syndrome (pigmentary glaucoma), scleritis, episcleritis choroidopathies (e.g., “White-dot” syndromes including, but not limited to, acute multifocal posterior placoid), retinopathies (e.g., cystoid macular edema, central serous choroidopathy and presumed ocular histoplasmosis syndrome, retinal vascular disease (e.g., diabetic retinopathy, Coat's disease and retinal arterial macroaneurysm), retinal artery occlusions, retinal vein occlusions, retinopathy of prematurity, retinitis pigmentosa, familial exudative vitreoretinopathy (FEVR), idiopathic polypoidal choroidal vasculopathy, epiretinal macular membranes and cataracts, and keratoconjunctivitis sicca (KCS).

In one embodiment, the ocular disorder is macular edema, Neovascular Glaucoma, diabetic retinopathy, or choroidal neovascularization. In another embodiment, the therapeutic agent is (i) Vascular Endothelial Growth Factor (VEGF) decoy, Pigment Derived Growth Factor (PDGF), Endostatin, Angiostatin, or Angiopoietin-1 or (ii) a nucleotide molecule coding for VEGF decoy, PDGF, Endostatin, Angiostatin, or Angiopoietin-1.

In one embodiment, the ocular disorder is macular degeneration. In another embodiment, the therapeutic agent is (i) VEGF decoy, PDGF, Endostatin, Angiostatin, Angiopoietin-1, or ATP Binding Cassette Subfamily A Member 4 or (ii) a nucleotide molecule coding for VEGF decoy, PDGF, Endostatin, Angiostatin, Angiopoietin-1, ATP Binding Cassette Subfamily A Member 4, glutamate agonist, or glutamate antagonist.

In one embodiment, the ocular disorder is ischemic optic neuropathy. In another embodiment, the therapeutic agent is (i) Allotopic NADH dehydrogenase Unit 4 or (ii) a nucleotide molecule coding for Allotopic NADH dehydrogenase Unit 4.

In one embodiment, the ocular disorder is a retinopathy. In another embodiment, the therapeutic agent is (i) Glial Cell Derived Neurotropic Factor or Peripherin-2 or (ii) a nucleotide molecule coding for Glial Cell Derived Neurotropic Factor or Peripherin-2.

In one embodiment, the ocular disorder is retinitis pigmentosa. In another embodiment, the therapeutic agent is (i) Retinal Pigment Specific 65 kDa protein or (ii) a nucleotide molecule coding for Retinal Pigment Specific 65 kDa protein or (iii) a source of electrical stimulation such as a quantum dot.

In one embodiment, the ocular disorder is a viral infection of the eye. In another embodiment, the therapeutic agent is an antisense oligonucleotide that inhibits viral replication. In another embodiment, the antisense oligonucleotide inhibits cytomegalovirus (CMV) replication.

In one embodiment, the peptide has Formula (I):

X—[NH—CHR¹—C(O)—NH—CHR²—C(O)]ₓ—Y (I)

or a pharmaceutically acceptable salt or tautomer thereof, wherein
R¹ is H or the side chain of a neutral amino acid;
R² is the side chain of a basic amino acid;
x is 2-5 inclusive;
X is —H or a residue of a therapeutic agent; and
Y is —OH, or a residue of a therapeutic agent; with the proviso that one of X or Y is a residue of a therapeutic agent.

In one embodiment, R¹ is CH₃ and R² is (imidazole-4-yl)methyl. In another embodiment, x is 2.

In one embodiment, the peptide has a Formula (II):

H—[NH—CHR³—C(O)—NH—CHR⁴—C(O)]ₓ—OH (II)

or a pharmaceutically acceptable sale or tautomer thereof, wherein
R³ is H or the side chain of a neutral amino acid;
R⁴ is

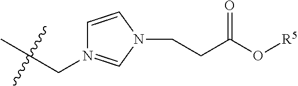

wherein R⁵ is a residue of a therapeutic agent;
x is 2-5 inclusive.

In one embodiment, the nucleotide molecule is part of an expression vector. In another embodiment, the nucleotide molecule has a sequence selected from the group consisting of SEQ ID NOS: 14, 18, 22, 26, 30, 34, 38, 42, 46, 50, 54, 58, 62, 66, 70, 74, 78, and 82.

In one embodiment, the therapeutic agent has an amino acid sequence selected from the group consisting of SEQ ID NOS: 15-17, 19-21, 23-25, 27-29, 31-33, 35-37, 39-41, 43-45, 47-49, 51-53, 55-57, 59-61, 63-65, 67-69, 71-73, 75-77, 79-81, 83-85, 87-89, and 91-93.

In one embodiment, the therapeutic agent is selected from the group consisting of acyclovir, betamethasone, dexamethasone, triamcinolone acetonide, bimatoprost, latanoprost, brinzolamide, carteolol, a fluoroquinolone (e.g., ciprofloxacin and ofloxacin), dexamethasone, dorzolamide, epinastine, fluorometholone, fusidic acid, gentamicin, levobunolol, lodoxamide, moxiflocin, nepaphenac, olopatadine, acetylcysteine, atropine, azithromycin, betaxolol, bromfenac, chloramphenicol, dictofenac, flurbiprofen, ganciclovir, homatropine, ketorolac, latanoprost, levofloxacin, loteprednol, nedocromil, rimexolone, timolol, travoprost, tafluprost, an aminoglycoside antibiotic (e.g., tobramycin), tropicamide, cyclosporine, fexofenadine, terfenadine, cetirizine, levocetirizine, desloratadine, hydroxyzine, a natural retinoid, and a synthetic retinoid.

In one embodiment, the nanostructure is a core surrounded by a shell, wherein the shell comprises at least two different molecules. In another embodiment, the nanostructure has a core with a diameter of from 1 to 10 nanometers. In another embodiment, the shell comprises two different molecules selected from the group consisting of ZnS, CdS, ZnSe and CdSe. In another embodiment, the nanostructure core comprises one or more molecules selected from group of molecules consisting of elements from columns II-IV, III-V or IV of the periodic table. In another embodiment, the nanostructure core comprises CdSe. In another embodiment, the nanostructure core comprises InP. In another embodiment, the shell comprises ZnS and CdS molecules. In another embodiment, the shell comprises from 1 to 10 monolayers. In another embodiment, a diameter of the nanostructure core is from 4 to 5 nanometers and the shell comprises from 3 to 6 monolayers. In another embodiment, the nanostructure core surrounded by the shell is annealed with ultraviolet radiation prior to and/or after attachment of the peptide to the surface of the shell.

In one embodiment, the nanoparticle composition is administered as part of a therapeutic composition. In another embodiment, the nanoparticle composition is administered topically to the eye. In another embodiment, the nanoparticle composition is administered by intravitreal administration.

In one embodiment, the nanostructures are quantum dots. In another embodiment, the quantum dots are capable of fluorescing.

In one embodiment, the peptide is reversibly linked to the therapeutic agent via a linkage that is capable of being cleaved.

In one embodiment, the quantum dot is capable of fluorescing and the linkage is capable of being cleaved by fluorescence emitted by the quantum dot, when the quantum dot is exposed to light.

In another embodiment, the therapeutic agent is also linked to a quenching agent such that fluorescence emitted by the quantum dot is quenched by the quenching agent, when the therapeutic agent is linked to the quantum dot.

In another embodiment, the linkage is pH labile. In another embodiment, the linkage is hydrolyzed at a pH less than 8.0. In another embodiment, the linkage is hydrolyzed at a pH of about 3.0 to about 6.0. In another embodiment, the linkage is enzymatically labile. In another embodiment, the linkage is enzymatically cleaved by a protease, an esterase, a hydrolase, a nuclease, a glycosidase, a lipase, a phosphatase, a sulfatase, or a phospholipase. In another embodiment, the linkage is enzymatically cleaved by a protease. In another embodiment, the protease is a trypsin-like protease. In another embodiment, the protease is a chymotrypsin-like protease. In another embodiment, the protease is an elastase-like protease. In another embodiment, the linkage is enzymatically cleaved by a hydrolase. In another embodiment, the hydrolase is an esterase.

In one embodiment, the peptide is reversibly linked to the therapeutic agent via a linkage that is capable of being cleaved by energy emitted by the quantum dot of a first wavelength, wherein upon exposure to light the quantum dot emits energy of a first wavelength when the therapeutic agent is linked, and emits energy of a second wavelength when the therapeutic agent has been released. In another embodiment, the quenching agent is conjugated to the peptide via a linkage that is enzymatically labile, wherein the quenching agent quenches the fluorescence of the quantum dot when the agent is linked to the quantum dot.

In one embodiment, the quantum dots further comprise a targeting molecule.

In one embodiment, the method further comprises exposing the nanoparticle to light sufficient to induce the quantum dot to emit energy, wherein the energy cleaves the linkage and the therapeutic agent is released.

### BRIEF DESCRIPTION OF THE DRAWINGS/FIGURES

FIG. 1 depicts a graph showing the concentration of SeeQ Cd/Se 655 Alt in rabbit vitreous following intravitreal injection of 168 pmole per eye. Data is expressed as mean±SD of 4 eyes.

FIG. 2 depicts a graph showing the concentration of SeeQ Cd/Se 655 Alt in rabbit retina following intravitreal injection of 168 pmole per eye. Data is expressed as mean±SD.

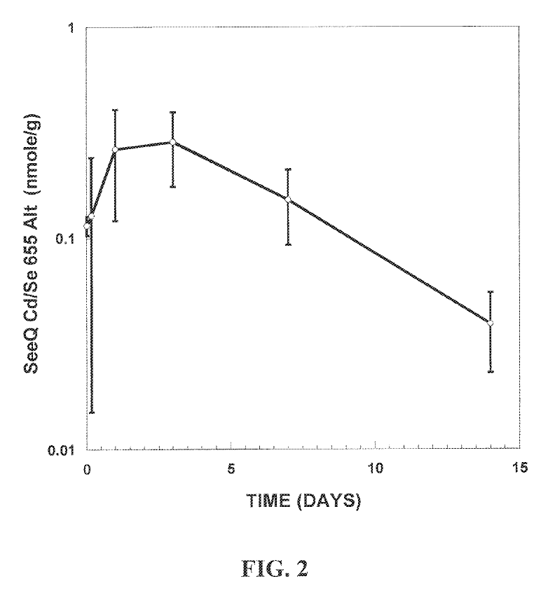

FIG. 3A depicts a method for making peptide-therapeutic agent conjugates.

FIG. 3B depicts a method for making peptide-therapeutic agent conjugates.

FIG. 4 depicts a method for making peptide therapeutic agent conjugates.

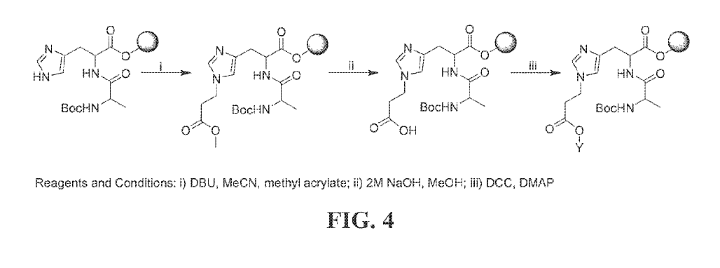

### DETAILED DESCRIPTION OF THE INVENTION

As used herein the specification, “a” or “an” may mean one or more. As used herein in the claim(s), when used in conjunction with the word “comprising,” the words “a” or “an” may mean one or more than one.

The term “targeted” as used herein encompasses the use of antigen-antibody binding, ligand-receptor binding, and other chemical and/or biochemical binding interactions to direct the binding of a chemical species to a specific site.

As used herein, “light” means electromagnetic radiation, which includes but is not limited to infrared, visible, and ultraviolet radiation. The wavelength of the light may be in the range of 600-2000 nm. In one embodiment, the light has a wavelength of 700-1200 nm. In another embodiment, the light has a wavelength of 750-1100 nm.

As used herein, a “core/shell” nanoparticle is a nanoparticle having a discrete core section surrounded by one or more shell layers.

As used herein, “nanoparticle” means one or more nanoparticles. As used herein, “core/shell nanoparticle” means one or more core/shell nanoparticles. As used herein, “shell” means one or more shells.

As used herein, “localized” means substantially limited to a desired area with only minimal, if any, dissemination outside of such area.

The nanoparticles may be administered to an animal using standard methods. Animals that may be treated include, but are not limited to, humans, non-human primates, cows, horses, pigs, dogs, cats, sheep, goats, rabbits, rats, mice, birds, chickens or fish.

“Nanometer” is 10⁻⁹ meter and is used interchangeably with the abbreviation “nm.”

A nanostructure has at least one region or characteristic dimension with a dimension of less than about 500 nm, and down to on the order of less than about 1 nm. The nanostructure may have any shape or morphology.

When referring to any numerical value, “about” means a value of ±10% of the stated value (e.g. “about 100 nm” encompasses a range of sizes from 90 nm to 110 nm, inclusive).

As used herein, the term “nanocrystal” refers to a nanostructure that is substantially monocrystalline. The terms “nanocrystal,” “nanodot,” “dot” and “quantum dot” are understood by the ordinarily skilled artisan to represent like structures and are used herein interchangeably. The present invention also encompasses the use of polycrystalline or amorphous nanocrystals. As used herein, the term “nanocrystal” also encompasses “luminescent nanocrystals.” As used herein, the term “luminescent nanocrystals” means nanocrystals that emit light when excited by an external energy source (suitably light).

Typically, the region of characteristic dimension will be along the smallest axis of the structure. Nanocrystals can be substantially homogenous in material properties, or in certain embodiments, can be heterogeneous. The optical properties of nanocrystals can be determined by their particle size, chemical or surface composition. In one embodiment, the luminescent nanocrystal size ranges between about 1 nm and about 15 nm.

Nanostructures for use herein can be produced using any method known to those skilled in the art. Suitable methods and exemplary nanocrystals are disclosed in Published U.S. patent application No. 2008/0237540; U.S. Pat. No. 7,374,807; U.S. patent application Ser. No. 10/796,832, filed Mar. 10, 2004; U.S. Pat. No. 6,949,206; and U.S. Provisional Patent Application No. 60/578,236, filed Jun. 8, 2004. The nanocrystals for use in the present invention can be produced from any suitable material, including an inorganic material, and more suitably an inorganic conductive or semiconductive material. Suitable materials include those disclosed in U.S. patent application Ser. No. 10/796,832, and include any type of semiconductor, including group II-VI, group group IV-VI and group IV semiconductors. Suitable semiconductor materials include, but are not limited to, Si, Ge, Sn, Se, Te, B, C (including diamond), P, BN, BP, BAs, AlN, AlP, AlAs, AlSb, GaN, GaP, GaAs, GaSb, InN, InP, InAs, InSb, ZnO, ZnS, ZnSe, ZnTe, CdS, CdSe, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCl, CuBr, CuI, Si₃N₄, Ge₃N₄, Al₂O₃, (Al, Ga, In)₂(S, Se, Te)₃, Al₂CO, and an appropriate combination of two or more such semiconductors.

In certain aspects, semiconductor nanocrystals may comprise a dopant from the group consisting of: a p-type dopant or an n-type dopant. The nanocrystals useful in the present invention can also comprise II-VI or semiconductors. Examples of II-VI or III-V semiconductor nanocrystals include any combination of an element from Group II, such as Zn, Cd and Hg, with any element from Group VI, such as S, Se, Te, Po, of the Periodic Table; and any combination of an element from Group III, such as B, Al, Ga, In, and Tl, with any element from Group V, such as N, P, As, Sb and Bi, of the Periodic Table.

The nanocrystals, including luminescent nanocrystals, useful in the present invention can also further comprise ligands conjugated, cooperated, associated or attached to their surface as described throughout. Suitable ligands include any group known to those skilled in the art, including those disclosed in U.S. Pat. No. 7,374,807, U.S. Pat. No. 6,949,206 and U.S. Provisional Patent Application No. 60/578,236.

In one embodiment, the peptide of Formula II can be synthesized from a peptide containing the basic amino acid sidechain (imidazol-4-yl)methyl (his), the method comprising:
i) reacting the peptide with methylacrylate in the presence of base;
ii) removing a methyl group from the methyl acrylate substituent by treatment with a base to expose a carboxylic acid group;
iii) coupling a therapeutic agent to the exposed carboxylic acid group with a coupling reagent, optionally in the presence of an additive.

Examples of base include, but are not limited to, 2,6-Di-tert-butylpyridine, N,N-diisopropylethylamine, 1,8-Diazabicyclo[5.4.0]undec-7-ene, sodium hydroxide, potassium hydroxide, and lithium hydroxide.

Examples of coupling reagents include, but are not limited to, dicyclohexylcarbodiimide (DCC), diisopropylcarbodiimide (DIC), and (N-(3-Dimethylaminopropyl)-N′-ethylcarbodiimide-HCl (EDAC).

Examples of additives include, but are not limited to, 1-Hydroxybenzotriazole (HOBt), hydroxy-3,4-dihydro-4-oxo-1,2,3-benzotriazine (HOOBt), N-hydroxysuccinimide (HOSu), 1-hydroxy-7-aza-1H-benzotriazole (HOAt), (4-(N,N-Dimethylamino)pyridine (DMAP).

In one embodiment, the peptide has Formula (I):

X—[NH—CHR¹—C(O)—NH—CHR²—C(O)]ₓ—Y (I)

or a pharmaceutically acceptable salt or tautomer thereof, wherein
R¹ is H or the side chain of a neutral amino acid;
R² is the side chain of a basic amino acid;
x is 2-5 inclusive;
X is —H or a residue of a therapeutic agent; and

Y is —OH, or a residue of a therapeutic agent. Examples of side chains of neutral amino acids include methyl (ala), isopropyl (val), 2-methylpropyl (leu), and 1-methylpropyl (ile).

Examples of side chains of basic amino acids include 4-aminobutyl (lys), 4-guanidinobutyl (arg) and (imidazol-4-yl)methyl (his).

Particular examples of peptides that may be linked to a therapeutic agent to give a compound of Formula (I) include, but are not limited to, ala-his, ala-his-ala-his (SEQ ID NO: 1), ala-his-ala-his-ala-his (SEQ ID NO: 2), ala-his-ala-his-ala-his-ala-his (SEQ ID NO: 3), gly-his, gly-his-gly-his (SEQ ID NO: 4), gly-his-gly-his-gly-his (SEQ ID NO: 5), gly-his-gly-his-gly-his-gly-his (SEQ ID NO: 6), aly-his-gly-his-gly-his-gly-his-gly-his (SEQ ID NO: 7), val-his, val-his-val-his (SEQ ID NO: 8), val-his-val-his-val-his (SEQ ID NO: 9), val-his-val-his-val-his-val-his (SEQ ID NO: 10), ile-his-ile-his (SEQ ID NO: 11), ile-his-ile-his-ile-his (SEQ ID NO: 12), and ile-his-ile-his-ile-his-ile-his (SEQ ID NO: 13).

Therapeutic agents that may be derivatized with a peptide include, without limitation, anti-inflammatories, anti-infectives, anti-virals, calcium channel blockers, neuroprotective agents, growth factors, growth factor antagonists, intraocular pressure lowering drugs, and antineoplastic drugs. Particular examples of therapeutic agents that are useful for the treatment of ocular disorders that may be derivatized with the peptide include acyclovir, betamethasone, bimatoprost, brinzolamide, carteolol, ciprofloxacin, dexamethasone, dorzolamide, epinastine, fluorometholone, fusidic acid, gentamicin, levobunolol, lodoxamide, moxifloxicin, nepaphenac, olopatadine, acetylcysteine, atropine, azithromycin, betaxolol, bromfenac, chloramphenicol, diclofenac, flurbiprofen, ganciclovir, homatropine, ketorolac, latanoprost, levofloxacin, loteprednol, nedocromil, ofloxacin, rimexolone, timolol, travoprost, tafluprost, tobramycin, tropicamide, cyclosporine, fexofenadine, terfenadine, cetirizine, levocetirizine, desloratadine, and hydroxyzine.

The derivatized therapeutic agents are exemplified by the following:

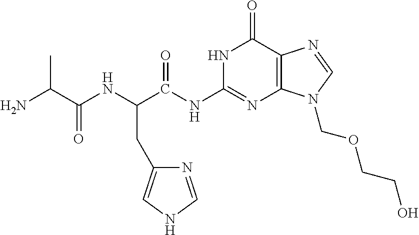 [SMILES: CC(N)C(=O)NC(Cc1c[nH]cn1)C(=O)Nc1nc2c(ncn2COCCO)c(=O)[nH]1]

### 2-(2-aminopropanamido)-N-(9-((2-hydroxyethoxy)methyl)-6-oxo-6,9-dihydro-1H-purin-2-yl)-3-(1H-imidazol-4-yl)propanamide

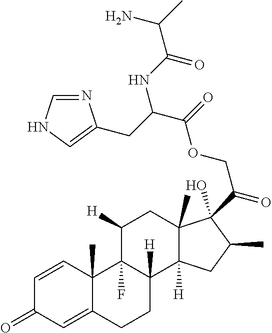

### 2-((8S,9R,10S,13S,14S,16S,17R)-9-fluoro-17-hydroxy-10,13,16-trimethyl-3-oxo-6,7,8,9,10,11,12,13,14,15,16,17-dodecahydro-3H-cyclopenta[a]phenanthren-17-yl)-2-oxoethyl alanylhistidinate

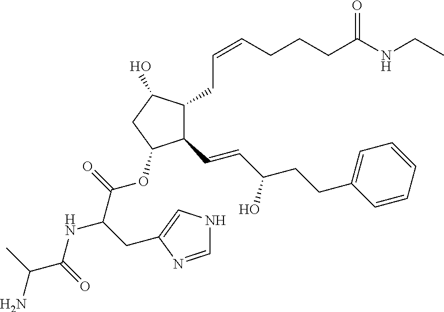 [SMILES: CCNC(=O)CCC/C=C\C[C@@H]1[C@@H](/C=C/[C@@H](O)CCc2ccccc2)[C@H](OC(=O)C(Cc2c[nH]cn2)NC(=O)C(C)N)C[C@@H]1O]

### (1R,2R,3R,4S)-3-((Z)-7-(ethylamino)-7-oxohept-2-en-1-yl)-4-hydroxy-2-((S,E)-3-hydroxy-5-phenylpent-1-en-1-yl)cyclopentyl alanylhistidinate

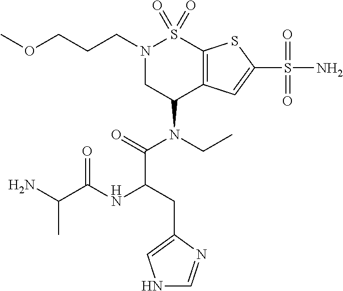

### 2-(2-aminopropanamido)-N-ethyl-3-(1H-imidazol-4-yl)-N—((R)-2-(3-methoxypropyl)-1,1-dioxido-6-sulfamoyl-3,4-dihydro-2H-thieno[3,2-e][1,2]thiazin-4-yl)propanamide

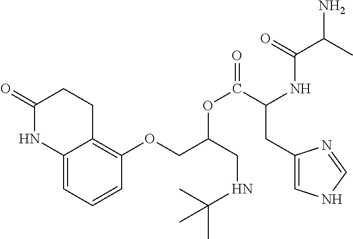 [SMILES: CC(N)C(=O)NC(Cc1c[nH]cn1)C(=O)OC(CNC(C)(C)C)COc1cccc2c1CCC(=O)N2]

### 1-(tert-butylamino)-3-((2-oxo-1,2,3,4-tetrahydroquinolin-5-yl)oxy)propan-2-yl alanylhistidinate

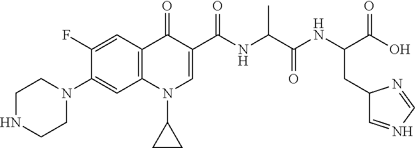 [SMILES: CC(NC(=O)c1cn(C2CC2)c2cc(N3CCNCC3)c(F)cc2c1=O)C(=O)NC(CC1C=NC=N1)C(=O)O]

### (1-cyclopropyl-6-fluoro-4-oxo-7-(piperazin-1-yl)-1,4-dihydroquinoline-3-carbonyl)alanylhistidine

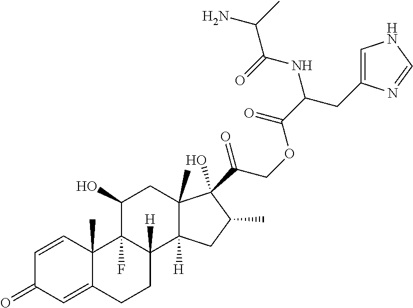

### 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethyl alanylhistidinate

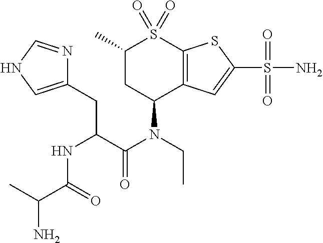 [SMILES: CCN(C(=O)C(Cc1c[nH]cn1)NC(=O)C(C)N)[C@H]1C[C@H](C)S(=O)(=O)c2sc(S(N)(=O)=O)cc21]

### 2-(2-aminopropanamido)-N-ethyl-3-(1H-imidazol-4-yl)-N-((4S,6S)-6-methyl-7,7-dioxido-2-sulfamoyl-5,6-dihydro-4H-thieno[2,3-b]thiopyran-4-yl)propanamide

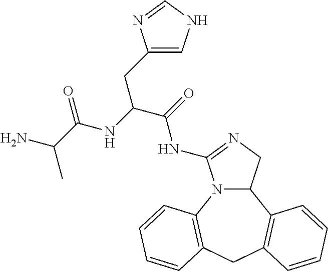 [SMILES: CC(N)C(=O)NC(Cc1c[nH]cn1)C(=O)NC1=NCC2c3ccccc3Cc3ccccc3N12]

### 2-(2-aminopropanamido)-N-(9,13b-dihydro-1H-dibenzo[c,f]imidazo[1,5-a]azepin-3-yl)-3-(1H-imidazol-4-yl)propanamide

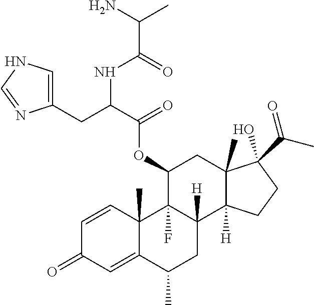

### (6S,8S,9R,10S,11S,13S,14S,17R)-17-acetyl-9-fluoro-17-hydroxy-6,10,13-trimethyl-3-oxo-6,7,8,9,10,11,12,13,14,15,16,17-dodecahydro-3H-cyclopenta[a]phenanthren-11-yl alanylhistidinate

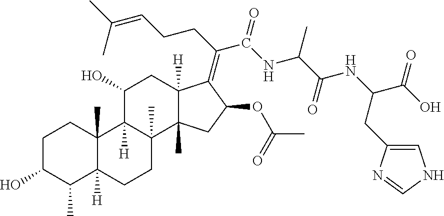

### ((Z)-2-((3R,4S,5S,8S,9S,10S,11R,13R,14S,16S)-16-acetoxy-3,11-dihydroxy-4,8,10,14-tetramethylhexadecahydro-17H-cyclopenta[a]phenanthren-17-ylidene)-6-methylhept-5-enoyl)alanylhistidine

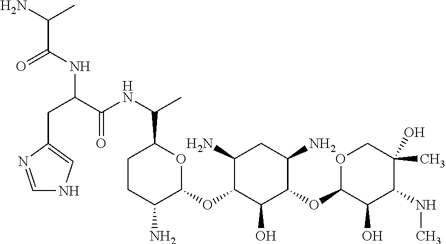 [SMILES: CN[C@@H]1[C@@H](O)[C@@H](O[C@@H]2[C@@H](O)[C@H](O[C@H]3O[C@H](C(C)NC(=O)C(Cc4c[nH]cn4)NC(=O)C(C)N)CC[C@H]3N)[C@@H](N)C[C@H]2N)OC[C@]1(C)O]

### N—((R)-1-((2S,5R,6R)-5-amino-6-(((1R,2S,3S,4R,6S)-4,6-diamine-3-(((2R,3R,4R,5R)-3,5-dihydroxy-5-methyl-4-(methylamino)tetrahydro-2H-pyran-2-yl)oxy)-2-hydroxycyclohexyl)oxy)tetrahydro-2H-pyran-2-yl)ethyl)-2-(2-aminopropanamido)-3-(1H-imidazol-4-yl)propanamide

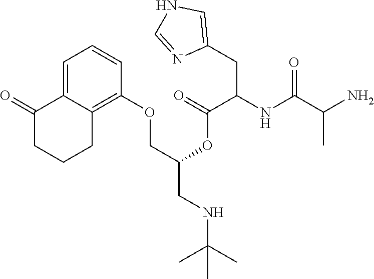 [SMILES: CC(N)C(=O)NC(Cc1c[nH]cn1)C(=O)O[C@H](CNC(C)(C)C)COc1cccc2c1CCCC2=O]

### (R)-1-(tert-butylamino)-3-((5-oxo-5,6,7,8-tetrahydronaphthalen-1-yl)oxy)propan-2-yl alanylhistidinate

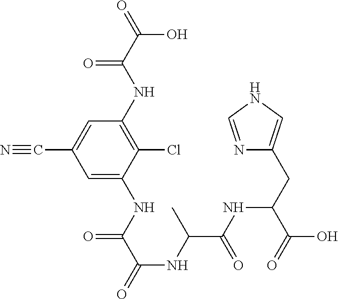 [SMILES: CC(NC(=O)C(=O)Nc1cc(C#N)cc(NC(=O)C(=O)O)c1Cl)C(=O)NC(Cc1c[nH]cn1)C(=O)O]

### (2-((3-(carboxyformamido)-2-chloro-5-cyanophenyl)amino)-2-oxoacetyl)alanylhistidine

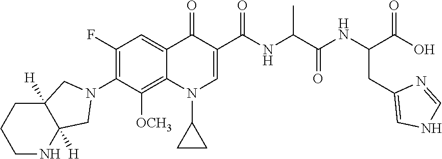 [SMILES: COc1c(N2C[C@@H]3CCCN[C@@H]3C2)c(F)cc2c(=O)c(C(=O)NC(C)C(=O)NC(Cc3c[nH]cn3)C(=O)O)cn(C3CC3)c12]

### (1-cyclopropyl-6-fluoro-8-methoxy-7-((4aS,7aS)-octahydro-6H-pyrrolo[3,4-b]pyridin-6-yl)-4-oxo-1,4-dihydroquinoline-3-carbonyl)alanylhistidine

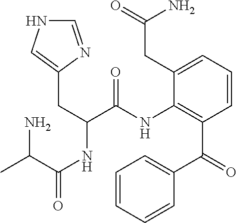 [SMILES: CC(N)C(=O)NC(Cc1c[nH]cn1)C(=O)Nc1c(CC(N)=O)cccc1C(=O)c1ccccc1]

### N-(2-(2-amino-2-oxoethyl)-6-benzoylphenyl)-2-(2-aminopropanamido)-3-(1H-imidazol-4-yl)propanamide

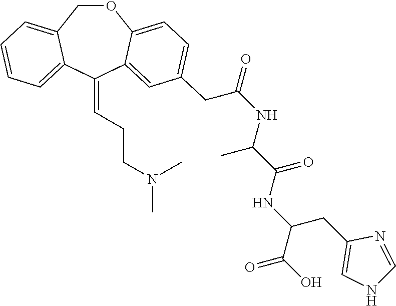 [SMILES: CC(NC(=O)Cc1ccc2c(c1)/C(=C\CCN(C)C)c1ccccc1CO2)C(=O)NC(Cc1c[nH]cn1)C(=O)O]

### (Z)-(2-(11-(3-(dimethylamino)propylidene)-6,11-dihydrodibenzo[b,e]oxepin-2-yl)acetyl)alanylhistidine

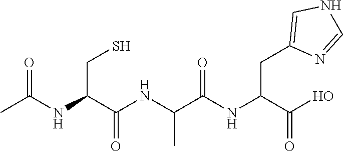 [SMILES: CC(=O)N[C@@H](CS)C(=O)NC(C)C(=O)NC(Cc1c[nH]cn1)C(=O)O]

### acetyl-L-cysteinylalanylhistidine

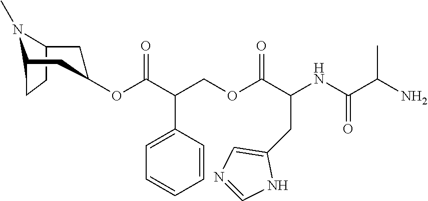

### 3-(((1R,3s,5S)-8-methyl-8-azabicyclo[3.2.1]octan-3-yl)oxy)-3-oxo-2-phenylpropyl alanylhistidinate

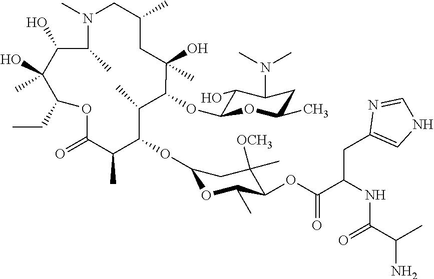

### (2S,3S,4R)-6-(((2R,3S,4R,5R,8R,10R,11R,12S,13S,14R)-11-(((2S,3R,4S,6R)-4-(dimethylamino)-3-hydroxy-6-methyltetrahydro-2H-pyran-2-yl)oxy)-2-ethyl-3,4,10-trihydroxy-3,5,6,8,10,12,14-heptamethyl-15-oxo-1-oxa-6-azacyclopentadecan-13-yl)oxy)-4-methoxy-2,4-dimethyltetrahydro-2H-pyran-3-yl alanylhistidinate

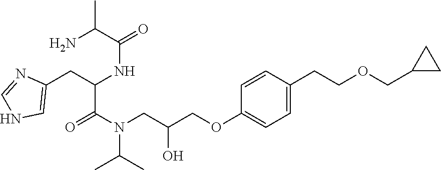 [SMILES: CC(N)C(=O)NC(Cc1c[nH]cn1)C(=O)N(CC(O)COc1ccc(CCOCC2CC2)cc1)C(C)C]

### 2-(2-aminopropanamido)-N-(3-(4-(2-(cyclopropylmethoxy)ethyl)phenoxy)-2-hydroxypropyl)-3-(1H-imidazol-4-yl)-N-isopropylpropanamide

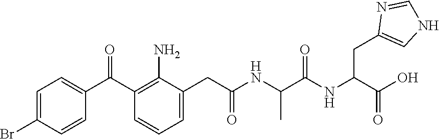 [SMILES: CC(NC(=O)Cc1cccc(C(=O)c2ccc(Br)cc2)c1N)C(=O)NC(Cc1c[nH]cn1)C(=O)O]

### (2-(2-amino-3-(4-bromobenzoyl)phenyl)acetyl)alanylhistidine

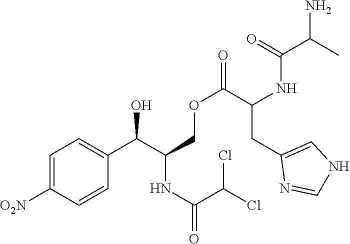 [SMILES: CC(N)C(=O)NC(Cc1c[nH]cn1)C(=O)OC[C@@H](NC(=O)C(Cl)Cl)[C@H](O)c1ccc([N+](=O)[O-])cc1]

### (2R,3R)-2-(2,2-dichloroacetamido)-3-hydroxy-3-(4-nitrophenyl)propyl alanylhistidinate

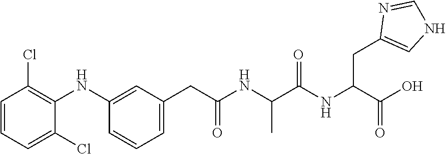 [SMILES: CC(NC(=O)Cc1cccc(Nc2c(Cl)cccc2Cl)c1)C(=O)NC(Cc1c[nH]cn1)C(=O)O]

### (2-(3-((2,6-dichlorophenyl)amino)phenyl)acetyl)alanylhistidine

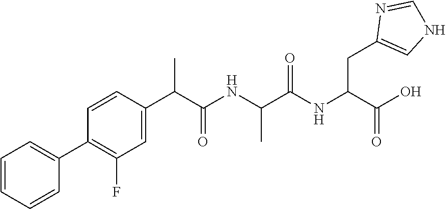 [SMILES: CC(NC(=O)C(C)c1ccc(-c2ccccc2)c(F)c1)C(=O)NC(Cc1c[nH]cn1)C(=O)O]

### (2-(2-fluoro-[1,1′-biphenyl]-4-yl)propanoyl)alanylhistidine

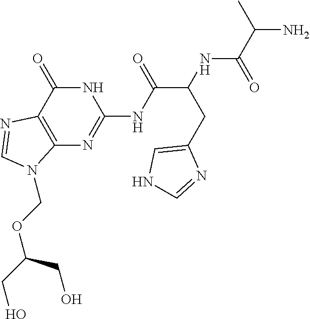

### 2-(2-aminopropanamido)-N-(9-(((1,3-dihydroxypropan-2-yl)oxy)methyl)-6-oxo-6,9-dihydro-1H-purin-2-yl)-3-(1H-imidazol-4-yl)propanamide

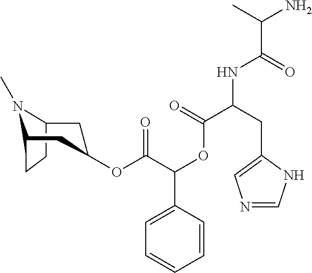

### 2-(((1R,3s,5S)-8-methyl-8-azabicyclo[3.2.1]octan-3-yl)oxy)-2-oxo-1-phenylethyl alanylhistidinate

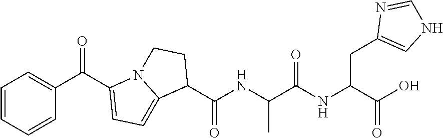 [SMILES: CC(NC(=O)C1CCn2c(C(=O)c3ccccc3)ccc21)C(=O)NC(Cc1c[nH]cn1)C(=O)O]

### (5-benzoyl-2,3-dihydro-1H-pyrrolizine-1-carbonyl)alanylhistidine

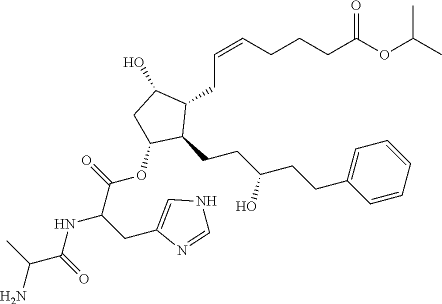 [SMILES: CC(C)OC(=O)CCC/C=C\C[C@@H]1[C@@H](CC[C@@H](O)CCc2ccccc2)[C@H](OC(=O)C(Cc2c[nH]cn2)NC(=O)C(C)N)C[C@@H]1O]

### isopropyl (Z)-7-((1R,2R,3R,5S)-3-((alanylhistidyl)oxy)-5-hydroxy-2-((R)-3-hydroxy-5-phenylpentyl)cyclopentyl)hept-5-enoate

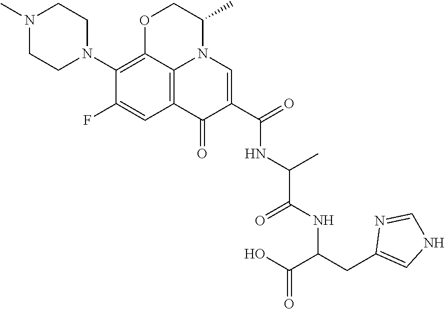 [SMILES: CC(NC(=O)c1cn2c3c(c(N4CCN(C)CC4)c(F)cc3c1=O)OC[C@@H]2C)C(=O)NC(Cc1c[nH]cn1)C(=O)O]

### ((S)-9-fluoro-3-methyl-10-(4-methylpiperazin-1-yl)-7-oxo-2,3-dihydro-7H-[1,4]oxazino[2,3,4-ij]quinoline-6-carbonyl)alanylhistidine

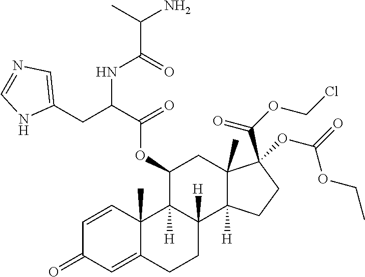

### chloromethyl (8S,9S,10R,11S,13S,14S,17R)-11-((alanylhistidyl)oxy)-17-((ethoxycarbonyl)oxy)-10,13-dimethyl-3-oxo-6,7,8,9,10,11,12,13,14,15,16,17-dodecahydro-3H-cyclopenta[a]phenanthrene-17-carboxylate

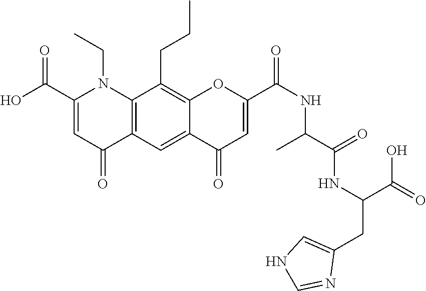 [SMILES: CCCc1c2oc(C(=O)NC(C)C(=O)NC(Cc3c[nH]cn3)C(=O)O)cc(=O)c2cc2c(=O)cc(C(=O)O)n(CC)c12]

### 2-((1-((1-carboxy-2-(1H-imidazol-4-yl)ethyl)amino)-1-oxopropan-2-yl)carbamoyl)-9-ethyl-4,6-dioxo-10-propyl-6,9-dihydro-4H-pyrano[3,2-g]quinoline-8-carboxylic acid

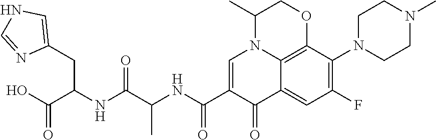 [SMILES: CC(NC(=O)c1cn2c3c(c(N4CCN(C)CC4)c(F)cc3c1=O)OCC2C)C(=O)NC(Cc1c[nH]cn1)C(=O)O]

### (9-fluoro-3-methyl-10-(4-methylpiperazin-1-yl)-7-oxo-2,3-dihydro-7H-[1,4]oxazino[2,3,4-ij]quinoline-6-carbonyl)alanylhistidine

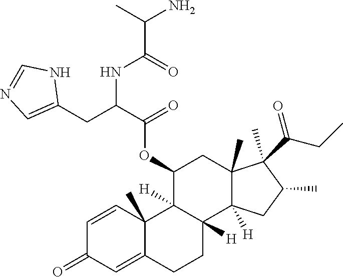 [SMILES: CCC(=O)[C@@]1(C)[C@H](C)C[C@H]2[C@@H]3CCC4=CC(=O)C=C[C@]4(C)[C@H]3[C@@H](OC(=O)C(Cc3cnc[nH]3)NC(=O)C(C)N)C[C@@]21C]

### (8S,9S,10R,11S,13S,14S,16R,17S)-10,13,16,17-tetramethyl-3-oxo-17-propionyl-6,7,8,9,10,11,12,13,14,15,16,17-dodecahydro-3H-cyclopenta[a]phenanthren-11-yl alanylhistidinate

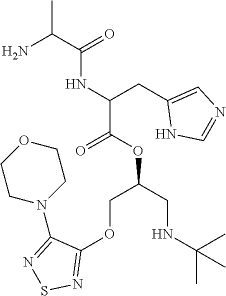 [SMILES: CC(N)C(=O)NC(Cc1cnc[nH]1)C(=O)O[C@@H](CNC(C)(C)C)COc1nsnc1N1CCOCC1]

### (S)-1-(tert-butylamino)-3-((4-morpholino-1,2,5-thiadiazol-3-yl)oxy)propan-2-yl alanylhistidinate

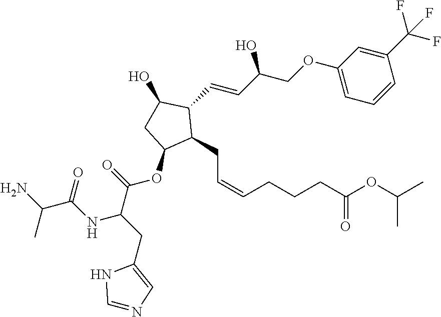 [SMILES: CC(C)OC(=O)CCC/C=C\C[C@@H]1[C@@H](/C=C/[C@@H](O)COc2cccc(C(F)(F)F)c2)[C@H](O)C[C@@H]1OC(=O)C(Cc1cnc[nH]1)NC(=O)C(C)N]

### isopropyl (Z)-7-((1R,2R,3R,5S)-5-((alanylhistidyl)oxy)-3-hydroxy-2-((R,E)-3-hydroxy-4-(3-(trifluoromethyl)phenoxy)but-1-en-1-yl)cyclopentyl)hept-5-enoate

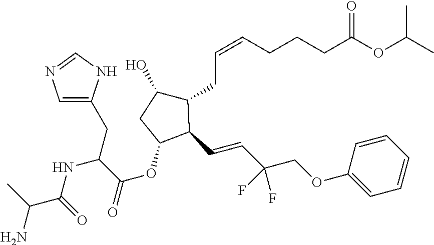 [SMILES: CC(C)OC(=O)CCC/C=C\C[C@@H]1[C@@H](/C=C/C(F)(F)COc2ccccc2)[C@H](OC(=O)C(Cc2cnc[nH]2)NC(=O)C(C)N)C[C@@H]1O]

### isopropyl (Z)-7-((1R,2R,3R,5S)-3-((alanylhistidyl)oxy)-2-((E)-3,3-difluoro-4-phenoxybut-1-en-1-yl)-5-hydroxycyclopentyl)hept-5-enoate

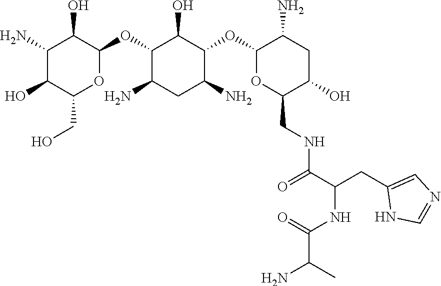 [SMILES: CC(N)C(=O)NC(Cc1cnc[nH]1)C(=O)NC[C@H]1O[C@H](O[C@H]2[C@H](O)[C@@H](O[C@H]3O[C@H](CO)[C@@H](O)[C@H](N)[C@H]3O)[C@H](N)C[C@@H]2N)[C@H](N)C[C@@H]1O]

### N-(((2R,3S,5R,6R)-5-amino-6-(((1R,2S,3S,4R,6S)-4,6-diamino-3-(((2S,3R,4S,5S,6R)-4-amino-3,5-dihydroxy-6-(hydroxymethyl)tetrahydro-2H-pyran-2-yl)oxy)-2-hydroxycyclohexyl)oxy)-3-hydroxytetrahydro-2H-pyran-2-yl)methyl)-2-(2-aminopropanamido)-3-(1H-imidazol-5-yl)propanamide

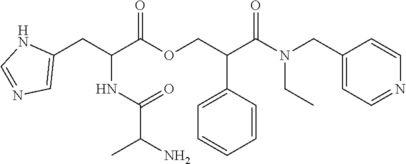 [SMILES: CCN(Cc1ccncc1)C(=O)C(COC(=O)C(Cc1cnc[nH]1)NC(=O)C(C)N)c1ccccc1]

### 3-(ethyl(pyridin-4-ylmethyl)amino)-3-oxo-2-phenylpropyl alanylhistidinate

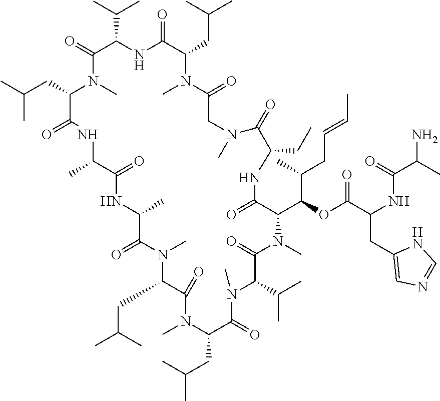 [SMILES: C/C=C/C[C@@H](C)[C@@H](OC(=O)C(Cc1cnc[nH]1)NC(=O)C(C)N)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### (1R,2R,E)-1-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-2-methylhex-4-en-1-yl alanylhistidinate

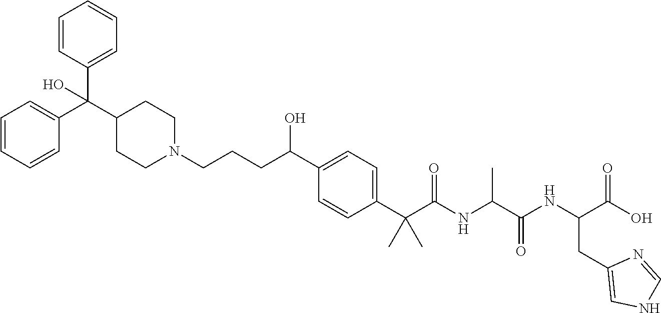 [SMILES: CC(NC(=O)C(C)(C)c1ccc(C(O)CCCN2CCC(C(O)(c3ccccc3)c3ccccc3)CC2)cc1)C(=O)NC(Cc1c[nH]cn1)C(=O)O]

### (2-(4-(1-hydroxy-4-(4-(hydroxydiphenylmethyl)piperidin-1-yl)butyl)phenyl)-2-methylpropanoyl)alanylhistidine

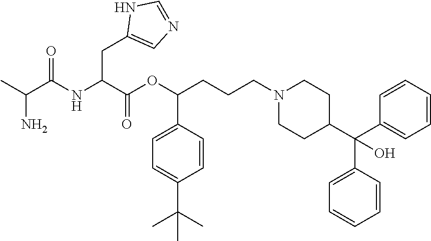 [SMILES: CC(N)C(=O)NC(Cc1cnc[nH]1)C(=O)OC(CCCN1CCC(C(O)(c2ccccc2)c2ccccc2)CC1)c1ccc(C(C)(C)C)cc1]

### 1-(4-(tert-butyl)phenyl)-4-(4-(hydroxydiphenylmethyl)piperidin-1-yl)butyl alanylhistidinate

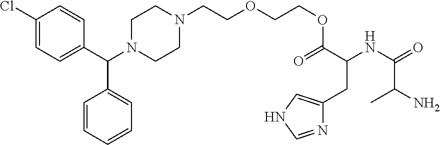 [SMILES: CC(N)C(=O)NC(Cc1c[nH]cn1)C(=O)OCCOCCN1CCN(C(c2ccccc2)c2ccc(Cl)cc2)CC1]

### 2-(2-(4-((4-chlorophenyl)(phenyl)methyl)piperazin-1-yl)ethoxy)ethyl alanylhistidinate

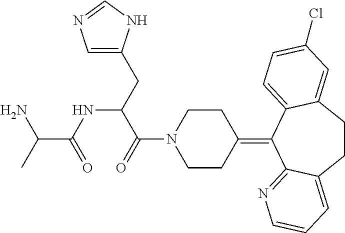 [SMILES: CC(N)C(=O)NC(Cc1cnc[nH]1)C(=O)N1CCC(=C2c3ccc(Cl)cc3CCc3cccnc32)CC1]

### 2-amino-N-(1-(4-(8-chloro-5,6-dihydro-11H-benzo[5,6]cyclohepta[1,2-b]pyridin-11-ylidene)piperidin-1-yl)-3-(1H-imidazol-5-yl)-1-oxopropan-2-yl)propanamide

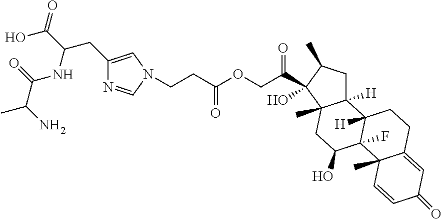

### Nα-alanyl-Nτ-(3-(2-((8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)-3-oxopropyl)histidine

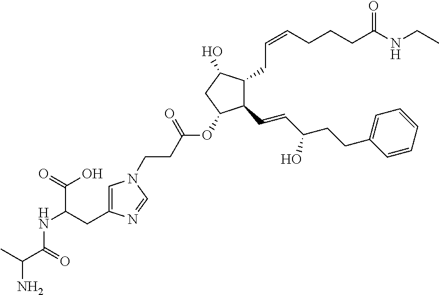 [SMILES: CCNC(=O)CCC/C=C\C[C@@H]1[C@@H](/C=C/[C@@H](O)CCc2ccccc2)[C@H](OC(=O)CCn2cnc(CC(NC(=O)C(C)N)C(=O)O)c2)C[C@@H]1O]

### Na-alanyl-Nt-(3-(((1R,2R,3R,4S)-3-((Z)-7-(ethylamino)-7-oxohept-2-en-1-yl)-4-hydroxy-2-((S,E)-3-hydroxy-5-phenylpent-1-en-1-yl)cyclopentyl)oxy)-3-oxopropyl)histidine

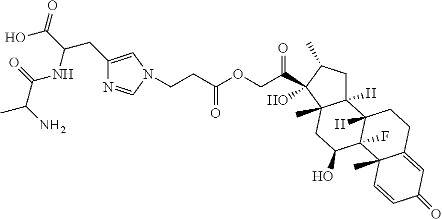

### Na-alanyl-Nt-(3-(2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl)-2-oxoethoxy)-3-oxopropyl)histidine

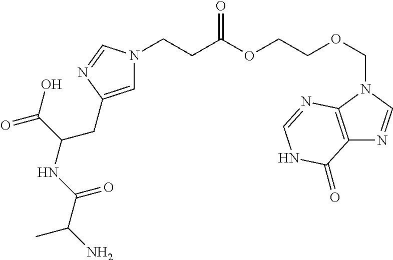 [SMILES: CC(N)C(=O)NC(Cc1cn(CCC(=O)OCCOCn2cnc3c(=O)[nH]cnc32)cn1)C(=O)O]

### Na-alanyl-Nt-(3-oxo-3-(2-((6-oxo-1,6-dihydro-9H-purin-9-yl)methoxy)ethoxy)propyl)histidine

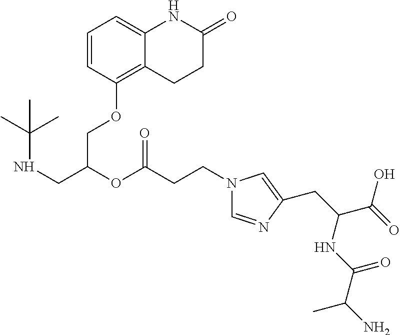 [SMILES: CC(N)C(=O)NC(Cc1cn(CCC(=O)OC(CNC(C)(C)C)COc2cccc3c2CCC(=O)N3)cn1)C(=O)O]

### Na-alanyl-Nt-(3-((1-(tert-butylamino)-3-((2-oxo-1,2,3,4-tetrahydroquinolin-5-yl)oxy)propan-2-yl)oxy)-3-oxopropyl)histidine

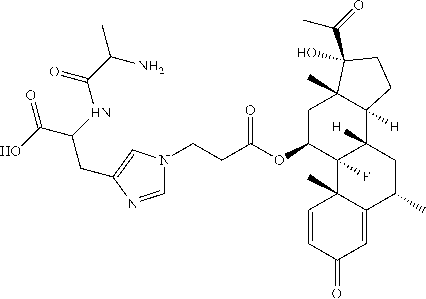

### Nt-(3-(((6S,8S,9R,10S,11S,13S,14S,17R)-17-acetyl-9-fluoro-17-hydroxy-6,10,13-trimethyl-3-oxo-6,7,8,9,10,11,12,13,14,15,16,17-dodecahydro-3H-cyclopenta[a]phenanthren-11-yl)oxy)-3-oxopropyl)-Na-alanylhistidine

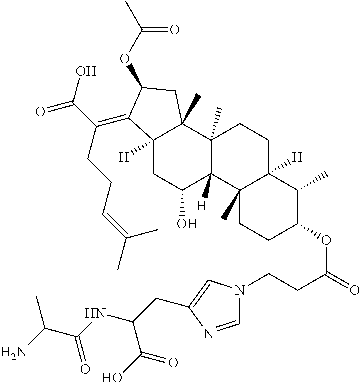 [SMILES: CC(=O)O[C@H]1C[C@@]2(C)[C@@H](C[C@@H](O)[C@H]3[C@@]4(C)CC[C@@H](OC(=O)CCn5cnc(CC(NC(=O)C(C)N)C(=O)O)c5)[C@@H](C)[C@@H]4CC[C@@]32C)/C1=C(\CCC=C(C)C)C(=O)O]

### (Z)-2-((3R,4S,5S,8S,9S,10S,11R,13R,14S,16S)-16-acetoxy-3-((3-(alanyl-Nt-histidino)propanoyl)oxy)-11-hydroxy-4,8,10,14-tetramethylhexadecahydro-17H-cyclopenta[a]phenanthren-17-ylidene)-6-methylhept-5-enoic acid

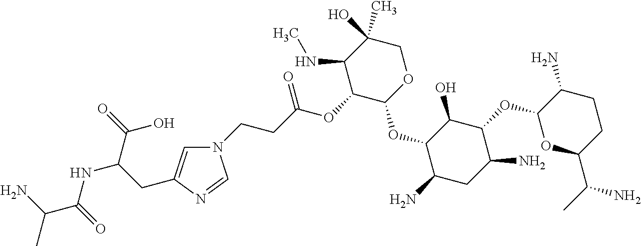 [SMILES: CN[C@@H]1[C@@H](OC(=O)CCn2cnc(CC(NC(=O)C(C)N)C(=O)O)c2)[C@@H](O[C@@H]2[C@@H](O)[C@H](O[C@H]3O[C@H]([C@@H](C)N)CC[C@H]3N)[C@@H](N)C[C@H]2N)OC[C@]1(C)O]

### Na-alanyl-Nt-(3-(((2R,3R,4R,5R)-2-(((1S,2S,3R,4S,6R)-4,6-diamino-3-(((2R,3R,6S)-3-amino-6-((R)-1-aminoethyl)tetrahydro-2H-pyran-2-yl)oxy)-2-hydroxycyclohexyl)oxy)-5-hydroxy-5-methyl-4-(methylamino)tetrahydro-2H-pyran-3-yl)oxy)-3-oxopropyl)histidine

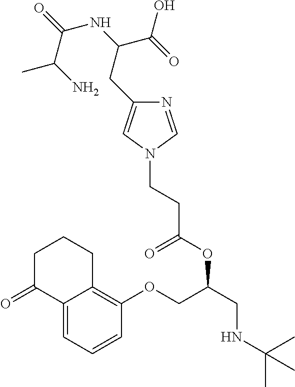 [SMILES: CC(N)C(=O)NC(Cc1cn(CCC(=O)O[C@@H](CNC(C)(C)C)COc2cccc3c2CCCC3=O)cn1)C(=O)O]

### Na-alanyl-Nt-(3-(((S)-1-(tert-butylamino)-3-((5-oxo-5,6,7,8-tetrahydronaphthalen-1-yl)oxy)propan-2-yl)oxy)-3-oxopropyl)histidine

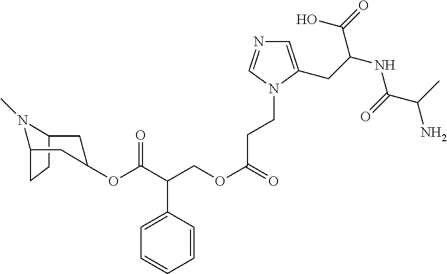 [SMILES: CC(N)C(=O)NC(Cc1cncn1CCC(=O)OCC(C(=O)OC1CC2CCC(C1)N2C)c1ccccc1)C(=O)O]

### Na-alanyl-Np-(3-(3-(((1R,3s,5S)-8-methyl-8-azabicyclo[3.2.1]octan-3-yl)oxy)-3-oxo-2-phenylpropoxy)-3-oxopropyl)histidine

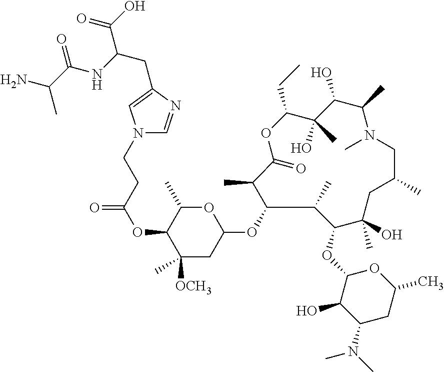 [SMILES: CC[C@H]1OC(=O)[C@H](C)[C@@H](OC2C[C@@](C)(OC)[C@@H](OC(=O)CCn3cnc(CC(NC(=O)C(C)N)C(=O)O)c3)[C@H](C)O2)[C@H](C)[C@@H](O[C@@H]2O[C@H](C)C[C@H](N(C)C)[C@H]2O)[C@](C)(O)C[C@@H](C)CN(C)[C@H](C)[C@@H](O)[C@]1(C)O]

### Na-alanyl-Nt-(3-(((2S,3S,4R)-6-(((2R,3S,4R,5R,8R,10R,11R,12S,13S,14R)-11-(((2S,3R,4S,6R)-4-(dimethylamino)-3-hydroxy-6-methyltetrahydro-2H-pyran-2-yl)oxy)-2-ethyl-3,4,10-trihydroxy-3,5,6,8,10,12,14-heptamethyl-15-oxo-1-oxa-6-azacyclopentadecan-13-yl)oxy)-4-methoxy-2,4-dimethyltetrahydro-2H-pyran-3-yl)oxy)-3-oxopropyl)histidine

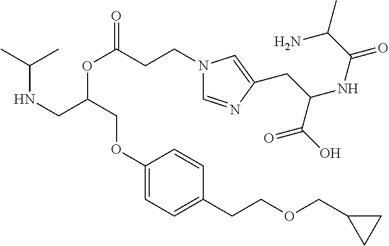 [SMILES: CC(C)NCC(COc1ccc(CCOCC2CC2)cc1)OC(=O)CCn1cnc(CC(NC(=O)C(C)N)C(=O)O)c1]

### Na-alanyl-Nt-(3-((1-(4-(2-(cyclopropylmethoxy)ethyl)phenoxy)-3-(isopropylamino)propan-2-yl)oxy)-3-oxopropyl)histidine

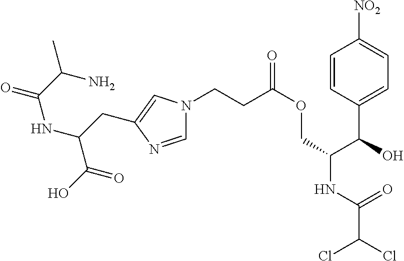 [SMILES: CC(N)C(=O)NC(Cc1cn(CCC(=O)OC[C@@H](NC(=O)C(Cl)Cl)[C@H](O)c2ccc([N+](=O)[O-])cc2)cn1)C(=O)O]

### Na-alanyl-Nt-(3-((2R,3R)-2-(2,2-dichloroacetamido)-3-hydroxy-3-(4-nitrophenyl)propoxy)-3-oxopropyl)histidine

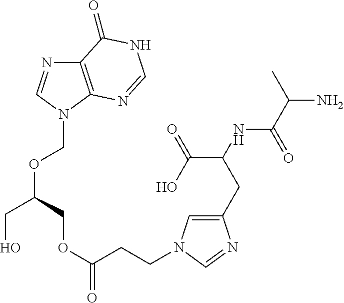 [SMILES: CC(N)C(=O)NC(Cc1cn(CCC(=O)OC[C@@H](CO)OCn2cnc3c(=O)[nH]cnc32)cn1)C(=O)O]

### Na-alanyl-Nt-(3-((R)-3-hydroxy-2-((6-oxo-1,6-dihydro-9H-purin-9-yl)methoxy)propoxy)-3-oxopropyl)histidine

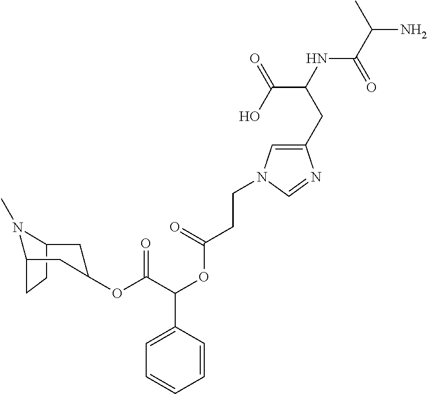 [SMILES: CC(N)C(=O)NC(Cc1cn(CCC(=O)OC(C(=O)OC2CC3CCC(C2)N3C)c2ccccc2)cn1)C(=O)O]

### Na-alanyl-Nt-(3-(2-(((1R,3s,5S)-8-methyl-8-azabicyclo[3.2.1]octan-3-yl)oxy)-2-oxo-1-phenylethoxy)-3-oxopropyl)histidine

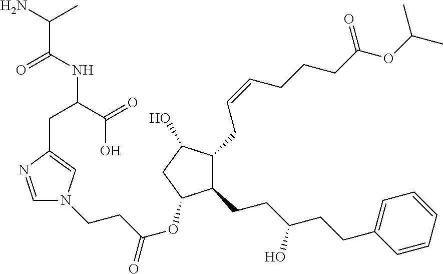 [SMILES: CC(C)OC(=O)CCC/C=C\C[C@@H]1[C@@H](CC[C@@H](O)CCc2ccccc2)[C@H](OC(=O)CCn2cnc(CC(NC(=O)C(C)N)C(=O)O)c2)C[C@@H]1O]

### Na-alanyl-Nt-(3-(((1R,2R,3R,4S)-4-hydroxy-2-((R)-3-hydroxy-5-phenylpentyl)-3-((Z)-7-isopropoxy-7-oxohept-2-en-1-yl)cyclopentyl)oxy)-3-oxopropyl)histidine

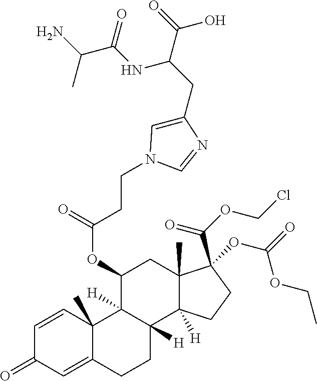 [SMILES: CCOC(=O)O[C@]1(C(=O)OCCl)CC[C@H]2[C@@H]3CCC4=CC(=O)C=C[C@]4(C)[C@H]3[C@@H](OC(=O)CCn3cnc(CC(NC(=O)C(C)N)C(=O)O)c3)C[C@@]21C]

### Na-alanyl-Nt-(3-(((8S,9S,10R,11S,13S,14S,17R)-17-((chloromethoxy)carbonyl)-17-((ethoxycarbonyl)oxy)-10,13-dimethyl-3-oxo-6,7,8,9,10,11,12,13,14,15,16,17-dodecahydro-3H-cyclopenta[a]phenanthren-11-yl)oxy)-3-oxopropyl)histidine

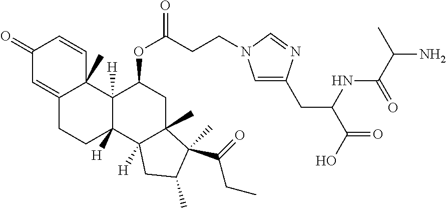 [SMILES: CCC(=O)[C@@]1(C)[C@H](C)C[C@H]2[C@@H]3CCC4=CC(=O)C=C[C@]4(C)[C@H]3[C@@H](OC(=O)CCn3cnc(CC(NC(=O)C(C)N)C(=O)O)c3)C[C@@]21C]

### Na-alanyl-Nt-(3-oxo-3-(((8S,9S,10R,11S,13S,14S,16R,17S)-10,13,16,17-tetramethyl-3-oxo-17-propionyl-6,7,8,9,10,11,12,13,14,15,16,17-dodecahydro-3H-cyclopenta[a]phenanthren-11-yl)oxy)propyl)histidine

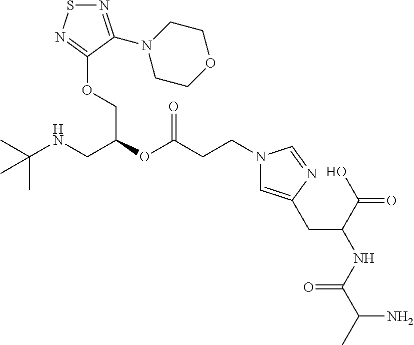 [SMILES: CC(N)C(=O)NC(Cc1cn(CCC(=O)O[C@@H](CNC(C)(C)C)COc2nsnc2N2CCOCC2)cn1)C(=O)O]

### Na-alanyl-Nt-(3-(((S)-1-(tert-butylamino)-3-((4-morpholino-1,2,5-thiadiazol-3-yl)oxy)propan-2-yl)oxy)-3-oxopropyl)histidine

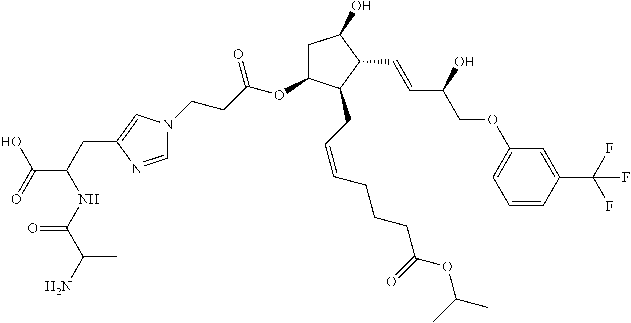 [SMILES: CC(C)OC(=O)CCC/C=C\C[C@@H]1[C@@H](/C=C/[C@@H](O)COc2cccc(C(F)(F)F)c2)[C@H](O)C[C@@H]1OC(=O)CCn1cnc(CC(NC(=O)C(C)N)C(=O)O)c1]

### Na-alanyl-Nt-(3-(((1S,2R,3R,4R)-4-hydroxy-3-((R,E)-3-hydroxy-4-(3-(trifluoromethyl)phenoxy)but-1-en-1-yl)-2-((Z)-7-isopropoxy-7-oxohept-2-en-1-yl)cyclopentyl)oxy)-3-oxopropyl)histidine

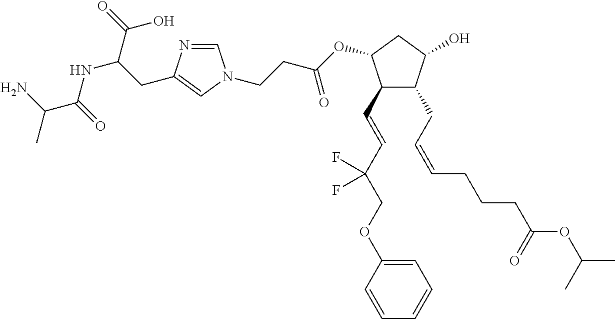 [SMILES: CC(C)OC(=O)CCC/C=C\C[C@@H]1[C@@H](/C=C/C(F)(F)COc2ccccc2)[C@H](OC(=O)CCn2cnc(CC(NC(=O)C(C)N)C(=O)O)c2)C[C@@H]1O]

### Na-alanyl-Nt-(3-(((1R,2R,3R,4S)-2-((E)-3,3-difluoro-4-phenoxybut-1-en-1-yl)-4-hydroxy-3-((Z)-7-isopropoxy-7-oxohept-2-en-1-yl)cyclopentyl)oxy)-3-oxopropyl)histidine

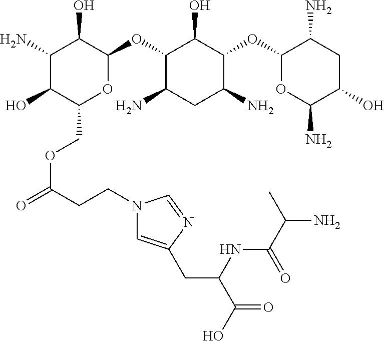 [SMILES: CC(N)C(=O)NC(Cc1cn(CCC(=O)OC[C@H]2O[C@H](O[C@@H]3[C@@H](O)[C@H](O[C@H]4O[C@H](N)[C@@H](O)C[C@H]4N)[C@@H](N)C[C@H]3N)[C@H](O)[C@@H](N)[C@@H]2O)cn1)C(=O)O]

### Na-alanyl-Nt-(3-(((2R,3S,4S,5R,6S)-4-amino-6-(((1S,2S,3R,4S,6R)-4,6-diamino-3-(((2S,3R,5S,6S)-3,6-diamino-5-hydroxytetrahydro-2H-pyran-2-yl)oxy)-2-hydroxycyclohexyl)oxy)-3,5-dihydroxytetrahydro-2H-pyran-2-yl)methoxy)-3-oxopropyl)histidine

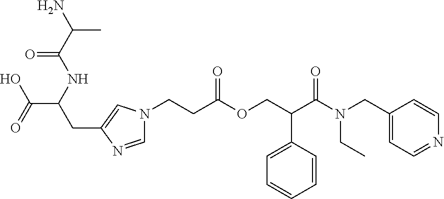 [SMILES: CCN(Cc1ccncc1)C(=O)C(COC(=O)CCn1cnc(CC(NC(=O)C(C)N)C(=O)O)c1)c1ccccc1]

### Na-alanyl-Nt-(3-(3-(ethyl(pyridin-4-yl)methyl)amino)-3-oxo-2-phenylpropoxy)-3-oxopropyl)histidine

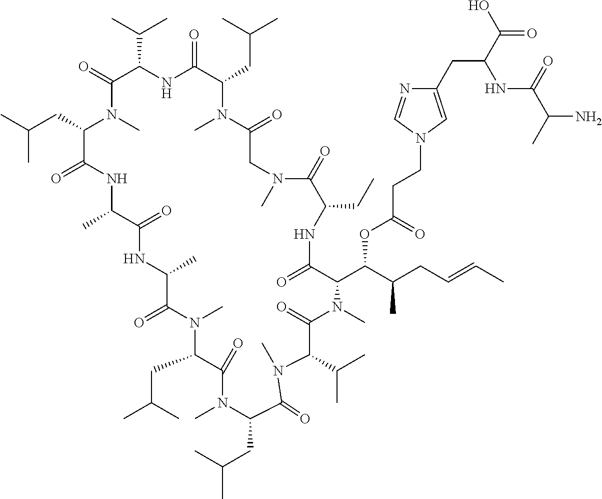 [SMILES: C/C=C/C[C@@H](C)[C@@H](OC(=O)CCn1cnc(CC(NC(=O)C(C)N)C(=O)O)c1)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### Na-alanyl-Nt-(3-(((1R,2R,E)-1-((2S,5S,11S,14S,17S,20S,23R,26S,29S,32S)-5-ethyl-11,17,26,29-tetraisobutyl-14,32-diisopropyl-1,7,10,16,20,23,25,28,31-nonamethyl-3,6,9,12,15,18,21,24,27,30,33-undecaoxo-1,4,7,10,13,16,19,22,25,28,31-undecaazacyclotritriacontan-2-yl)-2-methylhex-4-en-1-yl)oxy)-3-oxopropyl)histidine

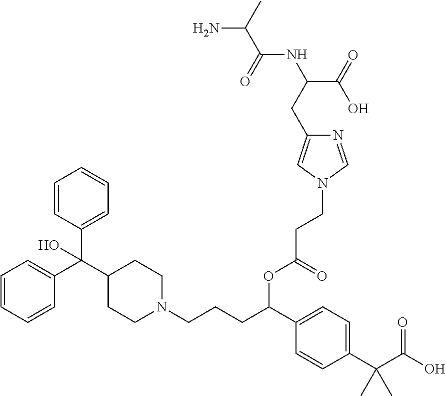 [SMILES: CC(N)C(=O)NC(Cc1cn(CCC(=O)OC(CCCN2CCC(C(O)(c3ccccc3)c3ccccc3)CC2)c2ccc(C(C)(C)C(=O)O)cc2)cn1)C(=O)O]

### 2-(4-(1-((3-(alanyl-Nt-histidino)propanoyl)oxy)-4-(4-(hydroxydiphenylmethyl)piperidin-1-yl)butyl)phenyl)-2-methylpropanoic acid

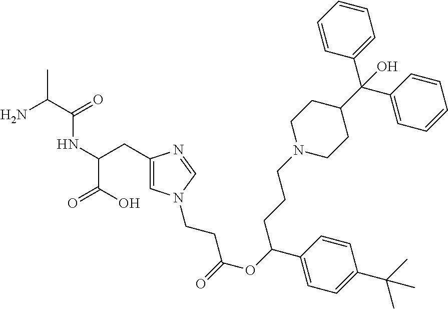 [SMILES: CC(N)C(=O)NC(Cc1cn(CCC(=O)OC(CCCN2CCC(C(O)(c3ccccc3)c3ccccc3)CC2)c2ccc(C(C)(C)C)cc2)cn1)C(=O)O]

### Na-alanyl-Nt-(3-(1-(4-(tert-butyl)phenyl)-4-(4-(hydroxydiphenylmethyl)piperidin-1-yl)butoxy)-3-oxopropyl)histidine

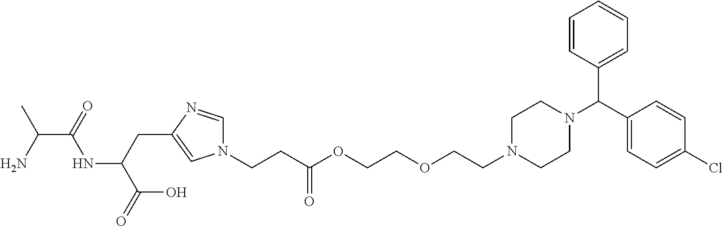 [SMILES: CC(N)C(=O)NC(Cc1cn(CCC(=O)OCCOCCN2CCN(C(c3ccccc3)c3ccc(Cl)cc3)CC2)cn1)C(=O)O]

### Na-alanyl-Nt-(3-(2-(2-(4-((4-chlorophenyl)(phenyl)methyl)piperazin-1-yl)ethoxy)ethoxy)-3-oxopropyl)histidine

In the alternative, the peptide may be conjugated to a protein therapeutic agent or a nucleotide molecule coding for the protein drug. Examples of protein drugs and nucleic acid molecules which may be used in the practice of the invention include, but are not limited to, those having the SEQ ID NOS: listed in the following table:

TABLE 1

| Sequence ID | Therapeutic Agent | Sequence Type |
|---|---|---|
| Seq ID NO: 14 | VEGF DECOY Homo sapien | Nucleotide Sequence |
| Seq ID NO: 15 | VEGF DECOY Protein-Ala-His | Amino Acid Sequence |
| Seq ID NO: 16 | VEGF DECOY Ala-His-Protein | Amino Acid Sequence |
| Seq ID NO: 17 | VEGF DECOY Homo sapien | Amino Acid Sequence |
| Seq ID NO: 18 | Pigment Derived Growth Factor Homo sapien | Nucleotide Sequence |
| Seq ID NO: 19 | Pigment Derived Growth Factor Protein-Ala-his | Amino Acid Sequence |
| Seq ID NO: 20 | Pigment Derived Growth Factor Ala-His-Protein | Amino Acid Sequence |
| Seq ID NO: 21 | Pigment Derived Growth Factor Homo sapien | Amino Acid Sequence |
| Seq ID NO: 22 | Pigment Derived Growth Factor Homo sapien | Nucleotide Sequence |
| Seq ID NO: 23 | Pigment Derived Growth Factor Protein-Ala-His | Amino Acid Sequence |
| Seq ID NO: 24 | Pigment Derived Growth Factor Ala-His-Protein | Amino Acid Sequence |
| Seq ID NO: 25 | Pigment Derived Growth Factor Homo sapien | Amino Acid Sequence |
| Seq ID NO: 26 | Pigment Derived Growth Factor Homo sapien | Nucleotide Sequence |
| Seq ID NO: 27 | Pigment Derived Growth Factor Protein-Ala-His | Amino Acid Sequence |
| Seq ID NO: 28 | Pigment Derived Growth Factor-Ala-His-Protein | Amino Acid Sequence |
| Seq ID NO: 29 | Pigment Derived Growth Factor Homo sapien | Amino Acid Sequence |
| Seq ID NO: 30 | Pigment Derived Growth Factor Homo sapien | Nucleotide Sequence |
| Seq ID NO: 31 | Pigment Derived Growth Factor Protein-Ala-His | Amino Acid Sequence |
| Seq ID NO: 32 | Pigment Derived Growth Factor Ala-His-Protein | Amino Acid Sequence |
| Seq ID NO: 33 | Pigment Derived Growth Factor Homo sapien | Amino Acid Sequence |
| Seq ID NO: 34 | Endostatin(HumanRecombinant) Homo sapien | Nucleotide Sequence |
| Seq ID NO: 35 | Endostatin(HumanRecombinant)Protein-Ala-His | Amino Acid Sequence |
| Seq ID NO: 36 | Endostatin(HumanRecombinant)Ala-His-Protein | Amino Acid Sequence |
| Seq ID NO: 37 | Endostatin(HumanRecombinant)Homo sapien | Amino Acid Sequence |
| Seq ID NO: 38 | Type XVIII Collagen Homo sapien | Nucleotide Sequence |
| Seq ID NO: 39 | Type XVIII Collagen Protein-Ala-His | Amino Acid Sequence |
| Seq ID NO: 40 | Type XVIII Collagen Ala-His-Protein | Amino Acid Sequence |
| Seq ID NO: 41 | Type XVIII Collagen Homo sapien | Amino Acid Sequence |
| Seq ID NO: 42 | Angiostatin Homo sapien | Nucleotide Sequence |
| Seq ID NO: 43 | Angiostatin Protein-Ala-His | Amino Acid Sequence |
| Seq ID NO: 44 | Angiostatin Ala-His-Protein | Amino Acid Sequence |
| Seq ID NO: 45 | Angiostatin Homo sapien | Amino Acid Sequence |
| Seq ID NO: 46 | Plasminogen Homo sapien | Nucleotide Sequence |
| Seq ID NO: 47 | Plasminogen Protein-Ala-His | Amino Acid Sequence |
| Seq ID NO: 48 | Plasminogen Ala-His-Protein | Amino Acid Sequence |
| Seq ID NO: 49 | Plasminogen Homo sapien | Amino Acid Sequence |
| Seq ID NO: 50 | Angiopoietin-1 Homo sapien | Nucleotide Sequence |
| Seq ID NO: 51 | Angiopoietin-1 Protein-Ala-His | Amino Acid Sequence |
| Seq ID NO: 52 | Angiopoietin-1 Ala-His-Protein | Amino Acid Sequence |
| Seq ID NO: 53 | Angiopoietin-1 Homo sapien | Amino Acid Sequence |
| Seq ID NO: 54 | Angiopoietin-1 Homo sapien | Nucleotide Sequence |
| Seq ID NO: 55 | Angiopoietin-1 Protein-Ala-His | Amino Acid Sequence |
| Seq ID NO: 56 | Angiopoietin-1 Ala-His-Protein | Amino Acid Sequence |
| Seq ID NO: 57 | Angiopoietin-1 Homo sapien | Amino Acid Sequence |
| Seq ID NO: 58 | Angiopoietin-1 Homo sapien | Nucleotide Sequence |
| Seq ID NO: 59 | Angiopoietin-1 Protein-Ala-His | Amino Acid Sequence |
| Seq ID NO: 60 | Angiopoietin-1 Ala-His-Protein | Amino Acid Sequence |
| Seq ID NO: 61 | Angiopoietin-1 Homo sapien | Amino Acid Sequence |
| Seq ID NO: 62 | ABCA4 Homo sapien | Nucleotide Sequence |
| Seq ID NO: 63 | ABCA4 Protein-Ala-His | Amino Acid Sequence |
| Seq ID NO: 64 | ABCA4 Ala-His-Protein | Amino Acid Sequence |
| Seq ID NO: 65 | ABCA4 Homo sapien | Amino Acid Sequence |
| Seq ID NO: 66 | NADH Dehydrogenase Unit4 Homo sapien | Nucleotide Sequence |
| Seq ID NO: 67 | NADH Dehydrogenase Unit4 Protein-Ala-His | Amino Acid Sequence |
| Seq ID NO: 68 | NADH Dehydrogenase Unit4 Protein-Ala-His | Amino Acid Sequence |
| Seq ID NO: 69 | NADH Dehydrogenase Unit4 Homo sapien | Amino Acid Sequence |
| Seq ID NO: 70 | GDNF Homo sapien | Nucleotide Sequence |
| Seq ID NO: 71 | GDNF Protein-Ala-His | Amino Acid Sequence |
| Seq ID NO: 72 | GDNF Ala-His-Protein | Amino Acid Sequence |
| Seq ID NO: 73 | GDNF Homo sapien | Amino Acid Sequence |
| Seq ID NO: 74 | GDNF Homo sapien | Nucleotide Sequence |
| Seq ID NO: 75 | GDNF Protein-Ala-His | Amino Acid Sequence |
| Seq ID NO: 76 | GDNF Ala-His-Protein | Amino Acid Sequence |
| Seq ID NO: 77 | GDNF Homo sapien | Amino Acid Sequence |
| Seq ID NO: 78 | GDNF Homo sapien | Nucleotide Sequence |
| Seq ID NO: 79 | GDNF Protein-Ala-His | Amino Acid Sequence |
| Seq ID NO: 80 | GDNF Ala-His-Protein | Amino Acid Sequence |
| Seq ID NO: 81 | GDNF Homo sapien | Amino Acid Sequence |
| Seq ID NO: 82 | GDNF Homo sapien | Nucleotide Sequence |
| Seq ID NO: 83 | GDNF Protein-Ala-HisProtein-Ala-His | Amino Acid Sequence |
| Seq ID NO: 84 | GDNF Protein-Ala-HisAla-His-Protein | Amino Acid Sequence |
| Seq ID NO: 85 | GDNF Homo sapien | Amino Acid Sequence |
| Seq ID NO: 86 | Peripherin-2 Homo sapien | Nucleotide Sequence |
| Seq ID NO: 87 | Peripherin-2 Protein-Ala-His | Amino Acid Sequence |
| Seq ID NO: 88 | Peripherin-2 Ala-His-Protein | Amino Acid Sequence |
| Seq ID NO: 89 | Peripherin-2 Homo sapien | Amino Acid Sequence |
| Seq ID NO: 90 | RPE65 Homo sapien | Nucleotide Sequence |
| Seq ID NO: 91 | RPE65 Protein-Ala-His | Amino Acid Sequence |
| Seq ID NO: 92 | RPE65 Ala-His-Protein | Amino Acid Sequence |
| Seq ID NO: 93 | RPE65 Homo sapien | Amino Acid Sequence |

In one embodiment, the therapeutic agent is an antisense oligonucleotide that inhibits viral replication. In another embodiment, the anti sense oligonucleotide inhibits cytomegalovirus (CMV) replication. Antisense oligonucleotides that are useful for the treatment of cytomegalovirus are disclosed in Henry et al., (2001).

When the therapeutic agent is a nucleotide molecule, it may be contained by a vector including plasmids, cosmids, artificial chromosomes, and modified viruses, as are known in the art. See, for example, Current Protocols in Molecular Biology (eds. Ausubel et al., Wiley, 2004 edition) and Molecular Cloning: A Laboratory Manual (Sambrook and Russell (Cold Spring Harbor Laboratory Press, 2001, third edition).

In one embodiment, the therapeutic agent is an antibody. In another embodiment, the antibody is bevacizumab (Avastin™) or ranibizumab (Lucentis™). In another embodiment, the ocular disorder is macular degeneration.

The nanoparticle composition may further comprise a targeting agent such as an antibody. The term antibody is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988). Antibody targeting agents which are expected to be useful in the eye include growth factors (e.g., VEGF and PDGF), growth factor receptors (e.g., VEGF arid PDGF), receptors of inflammatory mediators, and integrin receptors.

Monoclonal antibodies (MAbs) are recognized to have certain advantages, e.g., reproducibility and large-scale production. The antibodies may be of human, murine, monkey, rat, hamster, rabbit and chicken origin.

Humanized antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's disease are likewise known and such custom-tailored antibodies are also contemplated.

Antibodies may be further purified, if desired, using filtration, centrifugation and various chromatographic methods such as HPLC or affinity chromatography. Fragments of the antibodies can be obtained from the antibodies so produced by methods which include digestion with enzymes such as pepsin or papain, and/or by cleavage of disulfide bonds by chemical reduction. Alternatively, antibody fragments encompassed by the present invention can be synthesized using an automated peptide synthesizer or by expression of full-length gene or gene fragments in E. coli.

A molecular cloning approach may be used to generate monoclonal antibodies. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques is that many more antibodies can be produced and screened in a single round, and that new specificities are generated by H and L chain combination which further increases the chance of finding appropriate antibodies.

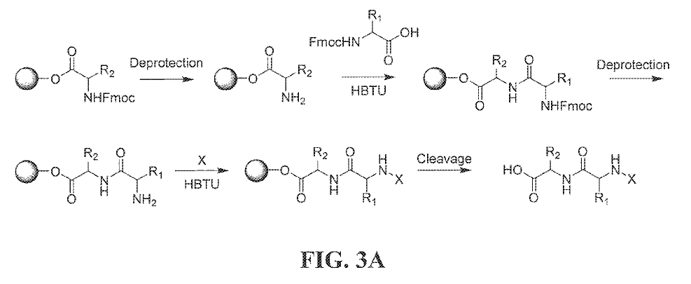

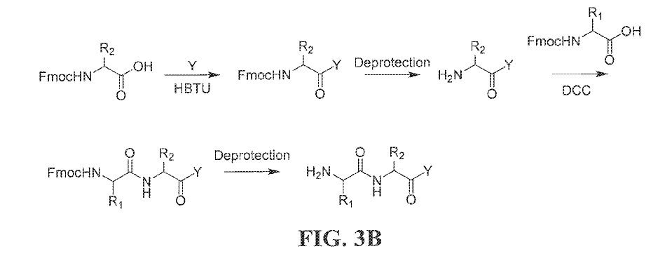

The peptide-therapeutic agent conjugates may be made by methods of solid phase synthesis exemplified by FIG. 3.

The nanoparticle compositions may be formulated with a pharmaceutically acceptable carrier for ophthalmic use. Particular carriers include saline, buffered saline, together with optional ingredients such as reduced glutathione, vitamin A, vitamin E. See U.S. Pat. No. 6,194,457.

The compositions may be administered by any means that achieves contact to the eye. In some embodiments, the composition is administered by intravitreal injection, eye drops, and the like. The location of the nanoparticle composition within the vitreous may be determine by ophthalmoscopy.

In one embodiment, the nanoparticle composition is exposed to light. In one embodiment, the method further comprises exposing the nanoparticle to light sufficient to induce the quantum dot to emit energy, wherein the energy cleaves the linkage and the therapeutic agent is released. In another embodiment, the wavelength of the light is in the range of 600-2000 nm. In another embodiment, the wavelength of the light is in the range of 700-1200 nm. In another embodiment, the wavelength of the light is in the range of 750-1100 nm. In a further embodiment, a laser provides the light to the nanoparticle.

Also provided is a method to tailor the physical/chemical properties of the nanoparticle to capitalize or the biological ocular environment to enhance contact time of therapeutic agents in different anatomical areas of the eye. In this embodiment, nanoparticles of different sizes and compositions are administered to different anatomical areas of the eye and the residence time within the eye is measured. For example, the nanoparticle compositions may be administered by injection into the vitreous body just outside of the lens, into the center of the vitreous body, on top of the retina, in the subconjunctival space, in subretinal space, or on top of the optic nerve, and the residence time measured to determine which compositions have the longest residence time when injected into a particular location.

### EXAMPLE

Intravitreal administration has been an effective way of delivering agents including drugs into the posterior chamber of the eye for the treatment of diseases such as macular degeneration (Kuppermann et al., 2007), diabetic retinopathy (Martidis et al., 2002) or viral infections (Henry et al., 2001). In most cases the agents have to be administered periodically in part due to the clearance from the vitreous and retina or due to enzymatic inactivation. Thus compounds that have slow clearance out of the vitreous or retina or longer residence in these tissues have the benefit of fewer administrations. In this experiment the clearance of SeeQ Cd/Se 655 Alt and its duration in the vitreous and retina was evaluated after intravitreal injection in rabbit eyes.

### Methods

New Zealand white rabbits weighing 2 to 2.5 kg were used in the study. Rabbits were anesthetized with intramuscular injection of 50 mg/kg of ketamine and 10 mg/kg xylazine. The eyes were topically anesthetized with proparacaine (0.5%) and the ocular surface was cleaned with providone iodine 0.5% before injection. Intravitreal injection was made 2 to 3 mm from the limbus in the superior quadrant of the globe. Twelve rabbits received single injection of 40 μl of SeeQ Cd/Se 655 Alt (4.2 μM aqueous solution, at pH 8) into both eyes, using 0.5 ml tuberculin syringe. Vitreous and retinal samples were collected at times 0, 4 hours, 1, 3, 7 and 14 days after injection. Two rabbits (4 eyes) were used per time point. Eyes were routinely examined for inflammation and toxicity using indirect ophthalmoscope and slit lamp. The localization of the drug in the vitreous was also assessed with indirect ophthalmoscope before the rabbits were sacrificed. Rabbits were euthanized with intravenous injection of ketamine and xylazine and eyes were enucleated. Animals that were not injected served as blank control. The vitreous and the retina samples were collected into pre-weighed tubes, weighed and frozen until they were analyzed. SeeQ Cd/Se 655 Alt concentration in the retina and vitreous were determined by measuring fluorescence (excitation 410 and emission 660). Samples were prepared, diluted and values measured from an external standard curve with concentrations of 40, 30, 20, 10, 5, 4, 3, 2, 1, and 0.5 nM. The limit of detection and limit of quantitation were 0.1 nM and 0.5 nM respectively.

### Results

Examination of the eyes during the two-week period showed no inflammation or any toxic effect of the drug. Examination of the posterior part of the eye using indirect ophthalmoscope and slit lamp showed the presence of the therapeutic in the vitreous. At time 0 it was located at the site of injection. Four hours after injection the drug was distributed in most of the vitreous humor and moved towards the retina. During the rest of the experimental periods (1 to 14 days), the presence of the drug was evident as seen by the orange color in the vitreous.

### Clearance of SeeQ Cd/Se 655 Alt

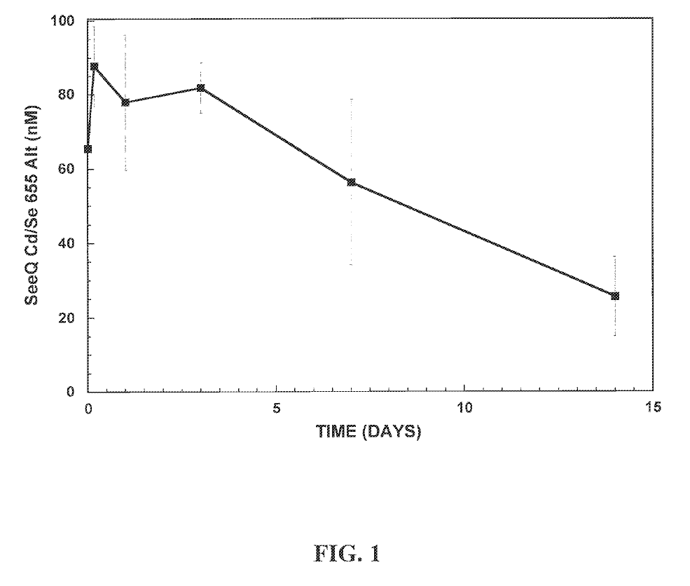

The concentration of SeeQ Cd/Se 655 Alt in the vitreous humor is shown in FIG. 1. The maximum concentration was constant in the first three days after injection with average of 80.9±10.7 nM, indicating very little clearance during this period. After day 7 and 14 vitreal concentration decreased to 56.4 nM (by 25%) and 25.6 nM (67%) respectively. The half-life of the clearance from the vitreous was 9 days. It appears to follow first order process.

In the retina the drug increased in the first three days reaching maximum in 1 day and remained high after 3 days. After day 7 and 14 retinal concentrations decreased to 0.152 nmoles/g (by 47%) and 0.0399 nmoles/g (88%) respectively. The half-life of the clearance from the retina was 7.5 days. The rate of clearance from the retina was also similar to that of the vitreous.

### Discussion

Intravitreal injection of drugs is a very effective way of targeted drug delivery to the posterior portion of the eye. In this experiment we showed the distribution and clearance of SeeQ Cd/Se 655 Alt in the vitreous and retina. After a single intravitreal injection, SeeQ Cd/Se 655 Alt was cleared from these tissues slowly with half-life of 7.5 days in the retina and 9 days in the vitreous. The concentration gradient created between the vitreous and retina allowed the retina to reach Cmax at 3 days after injection.

The rate of clearance of intravitreally administered material depends on the physicochemical properties of the material. These properties include lipophilicity, molecular size, structure, and surface charge of the material. In addition there are also active transport mechanisms, enzymatic degradation that can affect the residency and clearance of the drug. For example small molecule dexamethasone in a solution form disappears quickly (half-life of 3 days) (Berthe et al., 1992). On the other hand when it is prepared in a sustained release form (embedded in a lactic co-glycolic copolymer) it maintained a constant concentration for longer than one month (Chang-Lin et al., 2011). In this case a much higher concentration was accumulated in the retina. Unlike dexamethasone solution a suspension of triamcinolone acetonide had a different profile of clearance (Kim et al., 2006). The half-life of triamcinolone acetonide was 24 days for 4 mg and 39 days for 16 mg and the drug lasted for up to 4 to 6 months for the two doses administered (Kim et al., 2006). This long duration of triamcinolone is due to a very low solubility of the compound, and therefore the dissolution rate contributes to the steady state concentration in the retina. Other small molecules such as the hydrophilic antiviral foscarnet have a half-life of 12 hours (Kwak et al., 1994) Large protein molecules like Avastin™, an antibody that are used for the treatment of macular edema, had short half-life of 6 days but can be detected for longer than 30 days (Sinapis et al., 2011). This study also demonstrated that Avastin™ is delivered systemically as bevacizumab was found in the untreated contralateral eye.

In this experiment, SeeQ Cd/Se 655 Alt (168 pmoles/eye) was prepared in aqueous solution. In the retina and vitreous, the half-life was 7.5 and 9 days respectively. These values are 2.5 to three times longer than that reported for aqueous solution of dexamethasone sodium. Bioavailability of a material depends on the concentration present at the site of action. At present the physicochemical property of SeeQ Cd/Se 655 Alt in the rabbit vitreous is not known. However the observation that there was higher concentration of drug in the vitreous than in the retina at the end of two weeks suggests that the vitreous may act as a slow release depot.

### REFERENCES

Berthe P, Baudouin C, Garraffo R, et al. Toxicologic and pharmacokinetic analysis of intravitreal injections of foscarnet, either alone or in combination with ganciclovir. Invest. Ophthalmol. Vis. Sci 1994: 35:1018-1045.

Chang-Lin J-E, Burke J A, Peng Q. Pharmacokinetics of a Sustained-release Desamethasone Intravitreal Implant in Vitrectomized and Nonvitrectomized Eyes. Invest. Ophthamol. Vis. Sci. 52:4605-4609, 2011

Henry S P, Miner R C, Drew W L, et al. Antiviral activity and ocular kinetics of antisense oligonucleotides designed to inhibit CMV replication. Invest Ophthalmol Vis Sci. 42:2646-51, 2001

Kim H, Csaky K G, Gravlin L. Safety and pharmacokinetics of a Preservative-free Triamcinolone Acetonide Formulation for Intravitreal Administration. Retina, 26:523530, 2006

Kuppermann B D, Blumenkranz M S, Haller J A, et al. Randomized controlled study of an intravitreous dexamethasone drug delivery system in patients with persistent macular edema. Arch Ophthalmol. 125:309-317, 2007

Kwak H W, D'Amico D J. Evaluation of the retinal toxicity and pharmacokinetics of dexamethasone after intravitreal injection. Arch. Ophthalmol. 110:259-266, 1992

Martidis A, Duker J S, Greenberg P B, et al. Intravitreal triamcinolone for refractory diabetic macular edema. Ophthalmology, 109:920-7, 2002.

Sinapis, C I, Routsias, J G, et al. Pharmacokinetics of intravitreal bevacizumab (Avastin™) in rabbits. Clin. Pharmacol. 5:697-704, 2011.

Having now fully described this invention, it will be understood by those of ordinary skill in the art that the same can be performed within a wide and equivalent range of conditions, formulations, and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All patents, patent applications and publications cited herein are fully incorporated by reference.

1. A method of treating an ocular disorder, comprising contacting the eye of a subject in need thereof with an effective amount of a therapeutic nanoparticle composition, the therapeutic nanoparticle composition comprising:
(i) at least one population of nanostructures,
(ii) a peptide attached to the at least one population of nanostructures, wherein the peptide has Formula (I):
X—[NH—CHR¹—C(O)—NH—CHR²—C(O)]ₓ—Y (I)
or a pharmaceutically acceptable salt or tautomer thereof, wherein
R¹ is H or the side chain of a neutral amino acid;
R² is the side chain of a basic amino acid or R³;
x is 2-5 inclusive;
X is —H or a residue of the therapeutic agent;
Y is —OH, or a residue of the therapeutic agent;
R³ is: 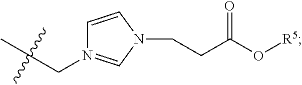
R⁵ is a residue of the therapeutic agent; and
provided that when R² is R³, X is —H, and Y is —OH, and
(iii) a therapeutic agent useful for the treatment of the ocular disorder attached to the at least one population of nanostructures or to the peptide.

2. The method of claim 1, wherein the therapeutic agent is selected from the group consisting of an anti-inflammatory, an anti-infective, an anti-viral, a calcium channel blocker, a neuroprotective agent, a growth factor, a growth factor antagonist, an intraocular pressure lowering drug, and an antineoplastic drug.

3. The method of claim 1, wherein the ocular disorder is selected from the group consisting of open angle glaucoma, angle closure glaucoma, aniridic glaucoma, congenital glaucoma, juvenile glaucoma, lens-induced glaucoma, neovascular glaucoma, post-traumatic glaucoma, steroid-induced glaucoma, Sturge-Weber syndrome glucoma, and uveitis-induced glaucoma, diabetic retinopathy, macular degeneration, choroidal neovascularization, vascular occlusion, vascular leak, retinal edema, bacterial conjunctivitis, fungal conjunctivitis, viral conjunctivitis, allergic conjunctivitis, uveitis, keratic precipitates, macular edema, inflammation response after intra-ocular lens implantation, uveitis syndromes, retinal vasculitis, sarcoidosis, Eales disease, acute retinal necrosis, Vogt Koyanaki Harada syndrome, ocular toxoplasmosis, radiation retinopathy, proliferative vitreoretinopathy, endophthalmitis, ocular glaucomas, optic neuritis, ischemic optic neuropathy, thyroid associated orbitopathy, orbital pseudotumor, pigment dispersion syndrome, scleritis, episcleritis choroidopathies, retinopathies, retinal vascular disease, retinal artery occlusions, retinal vein occlusions, retinopathy of prematurity, retinitis pigmentosa, familial exudative vitreoretinopathy (FEVR), idiopathic polypoidal choroidal vasculopathy, epiretinal macular membranes and cataracts, and keratoconjunctivitis sicca (KCS).

4. The method of claim 3, wherein the ocular disorder is macular edema, neovascular glaucoma, diabetic retinopathy, or choroidal neovascularization.

5. The method of claim 4, wherein the therapeutic agent is (i) Vascular Endothelial Growth Factor (VEGF) decoy, Pigment Derived Growth Factor (PDGF), Endostatin, Angiostatin, or Angiopoietin-1 or (ii) a nucleotide molecule coding for VEGF decoy, PDGF, Endostatin, Angiostatin, or Angiopoietin-1.

6. The method of claim 3, wherein the ocular disorder is macular degeneration.

7. The method of claim 6, wherein the therapeutic agent is (i) VEGF decoy, PDGF, Endostatin, Angiostatin, Angiopoietin-1, or ATP Binding Cassette Subfamily A Member 4 or (ii) a nucleotide molecule coding for VEGF decoy, PDGF, Endostatin, Angiostatin, Angiopoietin-1, ATP Binding Cassette Subfamily A Member 4, glutamate agonist, or glutamate antagonist.

8. The method of claim 3, wherein the ocular disorder is ischemic optic neuropathy.

9. The method of claim 8, wherein the therapeutic agent is (i) Allotopic NADH dehydrogenase Unit 4 or (ii) a nucleotide molecule coding for Allotopic NADH dehydrogenase Unit 4.

10. The method of claim 3, wherein the ocular disorder is a retinopathy.

11. The method of claim 10, wherein the therapeutic agent is (i) Glial Cell Derived Neurotropic Factor or Peripherin-2 or (ii) a nucleotide molecule coding for Glial Cell Derived Neurotropic Factor or Peripherin-2.

12. The method of claim 3, wherein the ocular disorder is retinitis pigmentosa.

13. The method of claim 12, wherein the therapeutic agent is (i) Retinal Pigment Specific 65 kDa protein or (ii) a nucleotide molecule coding for Retinal Pigment Specific 65 kDa protein or (iii) a source of electrical stimulation such as a quantum dot.

14. The method of claim 1, wherein the ocular disorder is a viral infection of the eye.

15. The method of claim 14, wherein the therapeutic agent is an antisense oligonucleotide that inhibits viral replication.

16. (canceled)

17. The method of claim 1, wherein R¹ is CH₃, R² is (imidazol-4-yl)methyl, and x is 2.

18. The method of claim 1, wherein the therapeutic agent is a nucleotide molecule that has a sequence selected from the group consisting of SEQ ID NOS: 14, 18, 22, 26, 30, 34, 38, 42, 46, 50, 54, 58, 62, 66, 70, 74, 78, and 82.

19. The method of claim 1, wherein the therapeutic agent has an amino acid sequence selected from the group consisting of SEQ ID NOS: 15-17, 19-21, 23-25, 27-29, 31-33, 35-37, 39-41, 43-45, 47-49, 51-53, 55-57, 59-61, 63-65, 67-69, 71-73, 75-77, 79-81, 83-85, 87-89, and 91-93.

20. The method of claim 1, wherein the therapeutic agent is selected from the group consisting of acyclovir, betamethasone, bimatoprost, brinzolamide, carteolol, ciprofloxacin, dexamethasone, triamcinolone acetonide, dorzolamide, epinastine, fluorometholone, fusidic acid, gentamicin, levobunolol, lodoxamide, moxiflocin, nepaphenac, olopatadine, acetylcysteine, atropine, azithromycin, betaxolol, bromfenac, chloramphenicol, diclofenac, flurbiprofen, ganciclovir, homatropine, ketorolac, latanoprost, levofloxacin, loteprednol, nedocromil, ofloxacin, rimexolone, timolol, travoprost, tafluprost, tobramycin, tropicamide, cyclosporine, fexofenadine, terfenadine, cetirizine, levocetirizine, desloratadine, and hydroxyzine.

21. The method of claim 1, wherein the nanostructure is a core surrounded by a shell, wherein the shell comprises at least two different molecules.

22. The method of claim 21, wherein the shell comprises two different molecules selected from the group consisting of ZnS, CdS, ZnSe and CdSe.

23. The method of claim 1, wherein the nanostructure comprises one or more molecules selected from group of molecules consisting of elements from columns II-IV, III-V or IV of the periodic table.

24. The method of claim 1, wherein the nanostructure comprises CdSe or InP.

25. The method of claim 21, wherein the diameter of the nanostructure core is from 4 to 5 nanometers and the shell comprises from 3 to 6 monolayers.

26. The method of claim 1, wherein the nanoparticle composition is administered by intravitreal administration.

27. The method of claim 1, wherein the nanostructures are quantum dots.

28. The method of claim 1, wherein the peptide is reversibly linked to the therapeutic agent by a linkage that is capable of being cleaved.

29. The method of claim 1, wherein the therapeutic nanoparticle composition is administered once every 1-4 weeks.

30. (canceled)


SEQUENCE LISTING (93 sequences)

SEQ ID NO: 1 - protein, 6 aa, Artificial Sequence
  artificial linkers
AHAHAH

SEQ ID NO: 2 - protein, 6 aa, Homo sapiens
AHAHAH

SEQ ID NO: 3 - protein, 8 aa, Artificial Sequence
  artificial linkers
AHAHAHAH

SEQ ID NO: 4 - protein, 4 aa, Artificial Sequence
  artificial linkers
GHGH

SEQ ID NO: 5 - protein, 6 aa, Artificial Sequence
  artificial linkers
GHGHGH

SEQ ID NO: 6 - protein, 8 aa, Artificial Sequence
  artificial linkers
GHGHGHGH

SEQ ID NO: 7 - protein, 10 aa, Artificial Sequence
  artificial linkers
GHGHGHGHGH

SEQ ID NO: 8 - protein, 4 aa, Artificial Sequence
  artificial linkers
VHVH

SEQ ID NO: 9 - protein, 6 aa, Artificial Sequence
  artificial linkers
VHVHVH

SEQ ID NO: 10 - protein, 8 aa, Artificial Sequence
  artificial linkers
VHVHVHVH

SEQ ID NO: 11 - protein, 4 aa, Artificial Sequence
  artificial linkers
IHIH

SEQ ID NO: 12 - protein, 6 aa, Artificial Sequence
  artificial linkers
IHIHIH

SEQ ID NO: 13 - protein, 8 aa, Artificial Sequence
  artificial linkers
IHIHIHIH

SEQ ID NO: 14 - DNA, 1578 bp, Homo sapiens
ggccgcccgtttgtggaaatgtatagcgaaattccggaaattattcatatgaccgaaggccgcgaactggtgattccgtgccgcgtgaccagcccgaacattaccgtgaccctgaaaaaatttccgctggataccctgattccggatggcaaacgcattatttgggatagccgcaaaggctttattattagcaacgcgacctataaagaaattggcctgctgacctgcgaagcgaccgtgaacggccatctgtataaaaccaactatctgacccatcgccagaccaacaccattattgatgtggtgctgagcccgagccatggcattgaactgagcgtgggcgaaaaactggtgctgaactgcaccgcgcgcaccgaactgaacgtgggcattgattttaactgggaatatccgagcagcaaacatcagcataaaaaactggtgaaccgcgatctgaaaacccagagcggcagcgaaatgaaaaaatttctgagcaccctgaccattgatggcgtgacccgcagcgatcagggcctgtatacctgcgcggcgagcagcggcctgatgaccaaaaaaaacagcacctttgtgcgcgtgcatgaaaaaccgtttgtggcgtttggcagcggcatggaaagcctggtggaagcgaccgtgggcgaacgcgtgcgcattccggcgaaatatctgggctatccgccgccggaaattaaatggtataaaaacggcattccgctggaaagcaaccataccattaaagcgggccatgtgctgaccattatggaagtgagcgaacgcgataccggcaactataccgtgattctgaccaacccgattagcaaagaaaaacagagccatgtggtgagcctggtggtgtatgtgccgccgggcccgggcgataaaacccatacctgcccgctgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaagcgaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 15 - protein, 528 aa, Artificial Sequence
  VEGF decoy Protein-Ala-His
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGKAH

SEQ ID NO: 16 - protein, 528 aa, Artificial Sequence
  VEGF DECOY Ala-His-Protein
AHGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 17 - protein, 526 aa, Homo sapiens
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 18 - DNA, 1599 bp, Homo sapiens
acgagacagtgatgcaatcacagaatccaaattgagtgcaggtcgctttaagaaaggagtagctgtaatctgaagcctgctggacgctggattagaaggcagcaaaaaaagctctgtgctggctggagccccctcagtgtgcaggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaacttctgttaaaaaaaaaaaaaaaaaa

SEQ ID NO: 19 - protein, 420 aa, Artificial Sequence
  Pigment Derived Growth Factor Protein- Ala-his
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGPAH

SEQ ID NO: 20 - protein, 420 aa, Artificial Sequence
  Pigment Derived Growth Factor Ala-His-Protein
AHMQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 21 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 22 - DNA, 1473 bp, Homo sapiens
tgggggcccagctgccagtgctggagacaagggccgtccgagatgaaccctttctgctgcctgccaagccactgggaggggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaacttctgtta

SEQ ID NO: 23 - protein, 420 aa, Artificial Sequence; identical to SEQ ID NO: 19
  Protein-Ala-His

SEQ ID NO: 24 - protein, 420 aa, Artificial Sequence; identical to SEQ ID NO: 20
  Ala-His-Protein

SEQ ID NO: 25 - protein, 418 aa, Homo sapiens; identical to SEQ ID NO: 21

SEQ ID NO: 26 - DNA, 1490 bp, Homo sapiens
acgagacagtgatgcaatcacagaatccaaattgagtgcaggtcgctttaagaaaggagtagctgtaatctgaagcctgctggacgctggattagaaggcagcaaaaaaagctctgtgctggctggagccccctcagtgtgcaggcttagagggactaggctgggtgtggagctgcagcgtatccacagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaacttctgtta

SEQ ID NO: 27 - protein, 233 aa, Artificial Sequence
  Protein-Ala-His
MKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGPAH

SEQ ID NO: 28 - protein, 233 aa, Artificial Sequence
  Ala-His-Protein
AHMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 29 - protein, 231 aa, Homo sapiens
MKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 30 - DNA, 1147 bp, Homo sapiens
ggcgcgatgtggggaaatctgctgcccccctggccagtgcctggggatgccagcagaagtcctggcaagtcacaggaagatgctggctgggaagtcagggcctgctgagcgctaaaccagaacccgagcctggcaggctctcaaagacgggatgcttgtcgtcgagtctcatacgctaacctctgctccgcctcttctcagagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaacttctgtta

SEQ ID NO: 31 - protein, 13 aa, Artificial Sequence
  Protein-Ala-His
FIGKILDPRGPAH

SEQ ID NO: 32 - protein, 13 aa, Artificial Sequence
  Ala-His-Protein
LLFIGKILDPRGP

SEQ ID NO: 33 - protein, 231 aa, Homo sapiens; identical to SEQ ID NO: 29

SEQ ID NO: 34 - DNA, 552 bp, Homo sapiens
atgcatagccatcgcgattttcagccggtgctgcatctggtggcgctgaacagcccgctgagcggcggcatgcgcggcattcgcggcgcggattttcagtgctttcagcaggcgcgcgcggtgggcctggcgggcacctttcgcgcgtttctgagcagccgcctgcaggatctgtatagcattgtgcgccgcgcggatcgcgcggcggtgccgattgtgaacctgaaagatgaactgctgtttccgagctgggaagcgctgtttagcggcagcgaaggcccgctgaaaccgggcgcgcgcatttttagctttgatggcaaagatgtgctgcgccatccgacctggccgcagaaaagcgtgtggcatggcagcgatccgaacggccgccgcctgaccgaaagctattgcgaaacctggcgcaccgaagcgccgagcgcgaccggccaggcgagcagcctgctgggcggccgcctgctgggccagagcgcggcgagctgccatcatgcgtatattgtgctgtgcattgaaaacagctttatgaccgcgagcaaa

SEQ ID NO: 35 - protein, 186 aa, Artificial Sequence
  Endostatin (Human Recombinant) Protein-Ala-His
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASKAH

SEQ ID NO: 36 - protein, 186 aa, Artificial Sequence
  Endostatin (Human Recombinant) Ala-His-Protein
AHMHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 37 - protein, 184 aa, Homo sapiens
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 38 - DNA, 6615 bp, Homo sapiens
agctccagccgcactgccccgatggctccctacccctgtggctgccacatcctgctgctgctcttctgctgcctggcggctgcccgggccaacctgctgaacctgaactggctttggttcaataatgaggacaccagccatgcagctaccacgatccctgagccccaggggcccctgcctgtgcagcccacagcagataccaccacacacgtgaccccccggaatggttccacagagccagcgacagcccctggcagccctgagccaccctcagagctgctggaagatggccaggacacccccacttctgccgagagcccggacgcgccagaggagaacattgccggtgtcggagccgagatcctgaacgtggccaaaggcatccggagcttcgtccagctgtggaatgacactgtccccactgagagcttggccagggcggaaaccctggtcctggagactcctgtgggcccccttgccctcgctgggccttccagcaccccccaggagaatgggaccactctctggcccagccgtggcattcctagctctccgggcgcccacacaaccgaggctggcaccttgcctgcacccaccccatcgcctccctccctgggcaggccctgggcaccactcacggggccctcagtgccaccaccatcttcaggtagagcttctctctcctccttgctgggcggggcccctccctggggaagcctgcaggacccagacagccaaggactctcgcccgccgcagccgctcccagccagcagctccaacgccctgacgtccgcctgcgcacgccacttctgcaccccctggtgatgggctccctgggcaagcacgcggccccctccgccttctcctctgggctcccgggcgcactgtctcaggtcgcagtcaccactttaaccagggacagcggtgcttgggtctcccacgtggctaactctgtggggccgggtcttgctaataactctgccctgctcggggctgaccccgaggcccccgccggtcgctgcctgcccctgccaccctccctgccagtctgcggccacctgggcatctcacgcttctggctgcccaaccacctccaccacgagagcggcgagcaggtgcgggccggggcacgggcgtgggggggcctgctgcagacgcactgccaccccttcctcgcctggttcttctgcctgctgctggtccccccatgcggcagcgtcccgccgcccgccccgccaccctgctgccagttctgcgaggccctgcaggatgcgtgttggagccgcctgggcgggggccggctgcccgtcgcctgtgcctcgctcccgacccaggaggatgggtactgtgtgctcattgggccggctgcagagcgcatcagcgaggaggtggggctgctgcagctccttggggaccccccgccccagcaggtcacccagacggatgaccccgacgtcgggctggcctacgtctttgggccagatgccaacagtggccaagtggcccggtaccacttccccagcctcttcttccgtgacttctcactgctgttccacatccggccagccacagagggcccaggggtgctgttcgccatcacggactcggcgcaggccatggtcttgctgggcgtgaagctctctggggtgcaggacgggcaccaggacatctccctgctctacacagaaccaggtgcaggccagacccacacagccgccagcttccggctccccgccttcgtcggccagtggacacacttagccctcagtgtggcaggtggctttgtggccctctacgtggactgtgaggagttccagagaatgccgcttgctcggtcctcacggggcctggagctggagcctggcgccgggctcttcgtggctcaggcggggggagcggaccctgacaagttccagggggtgatcgctgagctgaaggtgcgcagggacccccaggtgagccccatgcactgcctggacgaggaaggcgatgactcagatggggcatccggagactctggcagcgggctcggggacgcccgggagcttctcagggaggagacgggcgcggccctaaaacccaggctccccgcgccaccccccgtcaccacgccacccttggctggaggcagcagcacggaagattccagaagtgaagaagtcgaggagcagaccacggtggcttcgttaggagctcagacacttcctggctcagattctgtctccacgtgggacgggagtgtccggacccctgggggccgcgtgaaagagggcggcctgaaggggcagaaaggggagccaggtgttccgggcccacctggccgggcaggccccccaggatccccatgcctacctggtcccccgggtctcccgtgcccagtgagtcccctgggtcctgcaggcccagcgttgcaaactgtccccggaccacaaggacccccagggcctccggggagggacggcacccctggaagggacggcgagccgggcgaccccggtgaagacggaaagccgggcgacaccgggccacaaggcttccccgggactccaggggacgtaggtcccaagggcgacaagggagaccctggggttggagagagagggcccccaggaccccaagggcctccagggcccccaggaccctccttcagacacgacaagctgaccttcattgacatggagggatctggcttcgggggcgatctggaggccctgcggggtcctcgaggcttccctggacctcccggaccccccggtgtcccaggcctgcccggcgagccaggccgctttggggtgaacagctccgacgtcccaggacccgccggccttcctggtgtgcctgggcgcgagggtccccccgggtttcctggcctcccgggacccccaggccctccgggaagagaggggcccccaggaaggactgggcagaaaggcagcctgggtgaagcaggcgccccaggacataaggggagcaagggagcccccggtcctgctggtgctcgtggggagagcggcctggcaggagcccccggacctgctggaccaccaggcccccctgggccccctgggcccccaggaccaggactccccgctggatttgatgacatggaaggctccggggggcccttctggtcaacagcccgaagcgctgatgggccacagggacctcccggcctgccgggacttaagggggatcctggcgtgcctgggctgccgggggcgaagggagaagttggagcagatggagtccccgggttccccggcctccctggcagagagggcattgctgggccccaggggccaaagggagacagaggcagccggggagaaaagggagatccagggaaggacggagtcgggcagccgggcctccctggcccccccggacccccgggacctgtggtctacgtgtcggagcaggacggatccgtcctgagcgtgccgggacctgagggccggccgggtttcgcaggctttcccggacctgcaggacccaagggcaacctgggctctaagggcgaacgaggctccccgggacccaagggtgagaagggtgaaccgggcagcatcttcagccccgacggcggtgccctgggccctgcccagaaaggagccaagggagagccgggcttccgaggacccccgggtccatacggacggccggggtacaagggagagattggctttcctggacggccgggtcgccccgggatgaacggattgaaaggagagaaaggggagccgggagatgccagccttggatttggcatgaggggaatgcccggccccccaggacctccagggcccccaggccctccagggactcctgtttacgacagcaatgtgtttgctgagtccagccgccccgggcctccaggattgccagggaatcagggccctccaggacccaagggcgccaaaggagaagtgggcccccccggaccaccagggcagtttccgtttgactttcttcagttggaggctgaaatgaagggggagaagggagaccgaggtgatgcaggacagaaaggcgaaaggggggagcccgggggcggcggtttcttcggctccagcctgcccggcccccccggccccccaggcccacgtggctaccctgggattccaggtcccaagggagagagcatccggggccagcccggcccacctggacctcagggaccccccggcatcggctacgaggggcgccagggccctcccggccccccaggccccccagggcccccttcatttcctggccctcacaggcagactatcagcgttcccggccctccgggcccccctgggccccctgggccccctggaaccatgggcgcctcctcaggggtgaggctctgggctacacgccaggccatgctgggccaggtgcacgaggttcccgagggctggctcatcttcgtggccgagcaggaggagctctacgtccgcgtgcagaacgggttccggaaggtccagctggaggcccggacaccactcccacgagggacggacaatgaagtggccgccttgcagccccccgtggtgcagctgcacgacagcaacccctacccgcggcgggagcacccccaccccaccgcgcggccctggcgggcagatgacatcctggccagcccccctcgcctgcccgagccccagccctaccccggagccccgcaccacagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagccaccgcctggatgcggatggccggagaggaccggcggctcggaggaagcccccaccgtgggcagggagcggccggccagcccctggccccaggacctggctgccatactttcctgtatagttcacgtttcatgtaatcctcaagaaataaaaggaagccaaagagtgtatttttttaaaagtttaaaacagaagcctgatgctgacattcacctgccccaactctcccctgacctgtgagcccagctgggtcaggcagggtgcagtatcatgccctgtgcaacctcttggcctgatcagaccacggctcgatttctccaggatttcctgctttgggaagccgtgctcgccccagcaggtgctgacttcatctcccacctagcagcaccgttctgtgcacaaaacccagacctgttagcagacaggccccgtgaggcaatgggagctgaggccacactcagcacaaggccatctgggctcctccagggtgtgtgctcgccctgcggtagatgggagggaggctcaggtccctggggctagggggagccccttctgctcagctctgggccattctccacagcaaccccaggctgaagcaggttcccaagctcagaggcgcactgtgacccccagctccggcctgtcctccaacaccaagcacagcagcctggggctggcctcccaaatgagccatgagatgatacatccaaagcagacagctccaccctggccgagtccaagctgggagattcaagggacccatgagttggggtctggcagcctcccatccagggcccccatctcatgcccctggctgggacgtggctcagccagcacttgtccagctgagcgccaggatggaacacggccacatcaaagaggctgaggctggcacaggacatgcggtagccagcacacagggcagtgagggagggctgtcatctgtgcactgcccatggacaggctggctccagatgcagggcagtcattggctgtctcctaggaaacccatatccttaccctccttgggactgaaggggaaccccggggtgcccacaggccgccctgcgggtgaacaaagcagccacgaggtgcaacaaggtcctctgtcagtcacagccacccctgagatccggcaacatcaacccgagtcattcgttctgtggagggacaagtggactcagggcagcgccaggctgaccacagcacagccaacacgcacctgcctcaggactgcgacgaaaccggtggggctggttctgtaattgtgtgtgatgtgaagccaattcagacaggcaaataaaagtgaccttttacactgaaaaaaaaaaaaaaaaa

SEQ ID NO: 39 - protein, 1753 aa, Artificial Sequence
  Type XVIII collagen Protein-Ala-His
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASKAH

SEQ ID NO: 40 - protein, 1753 aa, Artificial Sequence
  Type XVIII collagen Ala-His-Protein
AHMAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 41 - protein, 1751 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 42 - DNA, 1164 bp, Homo sapiens
aaccgcaaaagcagcattattattcgcatgcgcgatgtggtgctgtttgaaaaaaaagtgtatctgagcgaatgcaaaaccggcaacggcaaaaactatcgcggcaccatgagcaaaaccaaaaacggcattacctgccagaaatggagcagcaccagcccgcatcgcccgcgctttagcccggcgacccatccgagcgaaggcctggaagaaaactattgccgcaacccggataacgatccgcagggcccgtggtgctataccaccgatccggaaaaacgctatgattattgcgatattctggaatgcgaagaagaatgcatgcattgcagcggcgaaaactatgatggcaaaattagcaaaaccatgagcggcctggaatgccaggcgtgggatagccagagcccgcatgcgcatggctatattccgagcaaatttccgaacaaaaacctgaaaaaaaactattgccgcaacccggatcgcgaactgcgcccgtggtgctttaccaccgatccgaacaaacgctgggaactgtgcgatattccgcgctgcaccaccccgccgccgagcagcggcccgacctatcagtgcctgaaaggcaccggcgaaaactatcgcggcaacgtggcggtgaccgtgagcggccatacctgccagcattggagcgcgcagaccccgcatacccataaccgcaccccggaaaactttccgtgcaaaaacctggatgaaaactattgccgcaacccggatggcaaacgcgcgccgtggtgccataccaccaacagccaggtgcgctgggaatattgcaaaattccgagctgcgatagcagcccggtgagcaccgaacagctggcgccgaccgcgccgccggaactgaccccggtggtgcaggattgctatcatggcgatggccagagctatcgcggcaccagcagcaccaccaccaccggcaaaaaatgccagagctggagcagcatgaccccgcatcgccatcagaaaaccccggaaaactatccgaacgcgggcctgaccatgaactattgccgcaacccggatgcggataaaggcccgtggtgctttaccaccgatccgagcgtgcgctgggaatattgcaacctgaaaaaatgcagcggcaccgaagcgagcgtggtggcgccgccgccg

SEQ ID NO: 43 - protein, 390 aa, Artificial Sequence
  Angiostatin Protein-Ala-His
NRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPAH

SEQ ID NO: 44 - protein, 390 aa, Artificial Sequence
  Angiostatin Ala-His-Protein
AHNRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPP

SEQ ID NO: 45 - protein, 388 aa, Homo sapiens
NRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPP

SEQ ID NO: 46 - DNA, 3538 bp, Homo sapiens
gaatcattaacttaatttgactatctggtttgtggatgcgtttactctcatgtaagtcaacaacatcctgggattgggacccactttctgggcactgctggccagtcccaaaatggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaattggacgggagacagagtgacgcactgactcacctagaggctggaacgtgggtagggatttagcatgctggaaataactggcagtaatcaaacgaagacactgtccccagctaccagctacgccaaacctcggcattttttgtgttattttctgactgctggattctgtagtaaggtgacatagctatgacatttgttaaaaataaactctgtacttaactttgatttgagtaaattttggttttggtcttcaacattttcatgctctttgttcaccccaccaatttttaaatgggcagatggggggatttagctgcttttgataaggaacagctgcacaaaggactgagcaggctgcaaggtcacagaggggagagccaagaagttgtccacgcatttacctcatcagctaacgagggcttgacatgcatttttactgtctttattcctgacactgagatgaatgttttcaaagctgcaacatgtatggggagtcatgcaaaccgattctgttattgggaatgaaatctgtcaccgactgcttgacttgagcccaggggacacggagcagagagctgtatatgatggagtgaaccggtccatggatgtgtaacacaagaccaactgagagtctgaatgttattctggggcacacgtgagtctaggattggtgccaagagcatgtaaatgaacaacaagcaaatattgaaggtggaccacttatttcccattgctaattgcctgcccggttttgaaacagtctgcagtacacacggtcacaggagaatgacctgtgggagagatacatgtttagaaggaagagaaaggacaaaggcacacgttttaccatttaaaatattgttaccaaacaaaaatatccattcaaaatacaatttaacaatgcaacagtcatcttacagcagagaaatgcagagaaaagcaaaactgcaagtgactgtgaataaagggtgaatgtagtctcaaatcctcaaa

SEQ ID NO: 47 - protein, 812 aa, Artificial Sequence
  Plasminogen  Protein-Ala-His
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNNAH

SEQ ID NO: 48 - protein, 812 aa, Artificial Sequence
  Plasminogen Ala-His-Protein
AHMEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 49 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 50 - DNA, 4367 bp, Homo sapiens
gccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 51 - protein, 500 aa, Artificial Sequence
  Angiopoietin-1 Protein-Ala-His
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFAH

SEQ ID NO: 52 - protein, 500 aa, Artificial Sequence
  Angiopoietin-1 Ala-His-Protein
AHMTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 53 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 54 - DNA, 4364 bp, Homo sapiens
gccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaagttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 55 - protein, 499 aa, Artificial Sequence
  Angiopoietin-1 Protein-Ala-His
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFAH

SEQ ID NO: 56 - protein, 500 aa, Artificial Sequence; identical to SEQ ID NO: 52
  Angiopoietin-1 Ala-His-Protein

SEQ ID NO: 57 - protein, 497 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 58 - DNA, 3549 bp, Homo sapiens
gtttttgtatttttagtagagacggggtttcaccatgtttgccaggatggtctcgatctcttgacctcgtgatccacccgccttggcctcccaaagtgctgggattacaggcgtgagccactgcgcccggcggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggtgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaagcaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggaaatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttaattgtttcagaataggataattaggtattcgaattttgtctttaaaattcatgtggtttctatgcaaagttcttcatatcatcacaacattatttgatttaaataaaattgaaagtaatatttgtgcaa

SEQ ID NO: 59 - protein, 300 aa, Artificial Sequence
  Angiopoietin-1 Protein-Ala-His
MEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFAH

SEQ ID NO: 60 - protein, 300 aa, Artificial Sequence
  Angiopoietin-1 Ala-His-Protein
AHMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 61 - protein, 298 aa, Homo sapiens
MEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 62 - DNA, 7326 bp, Homo sapiens
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 63 - protein, 2275 aa, Artificial Sequence
  ABCA4 Protein-Ala-His
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDAH

SEQ ID NO: 64 - protein, 2275 aa, Artificial Sequence
  ABCA4 Ala-His-Protein
AHMGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 65 - protein, 2273 aa, Homo sapiens [sequence omitted]

SEQ ID NO: 66 - DNA, 1378 bp, Homo sapiens
atgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 67 - protein, 461 aa, Artificial Sequence
  NADH dehydrogenase unit 4 Protein-Ala-His
MLKLIVPTIMLLPLTWLSKKHMIWINTTTHSLIISIIPLLFFNQINNNLFSCSPTFSSDPLTTPLLMLTTWLLPLTIMASQRHLSSEPLSRKKLYLSMLISLQISLIMTFTATELIMFYIFFETTLIPTLAIITRWGNQPERLNAGTYFLFYTLVGSLPLLIALIYTHNTLGSLNILLLTLTAQELSNSWANNLMWLAYTMAFMVKMPLYGLHLWLPKAHVEAPIAGSMVLAAVLLKLGGYGMMRLTLILNPLTKHMAYPFLVLSLWGMIMTSSICLRQTDLKSLIAYSSISHMALVVTAILIQTPWSFTGAVILMIAHGLTSSLLFCLANSNYERTHSRIMILSQGLQTLLPLMAFWWLLASLANLALPPTINLLGELSVLVTTFSWSNITLLLTGLNMLVTALYSLYMFTTTQWGSLTHHINNMKPSFTRENTLMFMHLSPILLLSLNPDIITGFSSAH

SEQ ID NO: 68 - protein, 461 aa, Artificial Sequence
  NADH dehydrogenase unit 4 Ala-His-Protein
AHMLKLIVPTIMLLPLTWLSKKHMIWINTTTHSLIISIIPLLFFNQINNNLFSCSPTFSSDPLTTPLLMLTTWLLPLTIMASQRHLSSEPLSRKKLYLSMLISLQISLIMTFTATELIMFYIFFETTLIPTLAIITRWGNQPERLNAGTYFLFYTLVGSLPLLIALIYTHNTLGSLNILLLTLTAQELSNSWANNLMWLAYTMAFMVKMPLYGLHLWLPKAHVEAPIAGSMVLAAVLLKLGGYGMMRLTLILNPLTKHMAYPFLVLSLWGMIMTSSICLRQTDLKSLIAYSSISHMALVVTAILIQTPWSFTGAVILMIAHGLTSSLLFCLANSNYERTHSRIMILSQGLQTLLPLMAFWWLLASLANLALPPTINLLGELSVLVTTFSWSNITLLLTGLNMLVTALYSLYMFTTTQWGSLTHHINNMKPSFTRENTLMFMHLSPILLLSLNPDIITGFSS

SEQ ID NO: 69 - protein, 459 aa, Homo sapiens
MLKLIVPTIMLLPLTWLSKKHMIWINTTTHSLIISIIPLLFFNQINNNLFSCSPTFSSDPLTTPLLMLTTWLLPLTIMASQRHLSSEPLSRKKLYLSMLISLQISLIMTFTATELIMFYIFFETTLIPTLAIITRWGNQPERLNAGTYFLFYTLVGSLPLLIALIYTHNTLGSLNILLLTLTAQELSNSWANNLMWLAYTMAFMVKMPLYGLHLWLPKAHVEAPIAGSMVLAAVLLKLGGYGMMRLTLILNPLTKHMAYPFLVLSLWGMIMTSSICLRQTDLKSLIAYSSISHMALVVTAILIQTPWSFTGAVILMIAHGLTSSLLFCLANSNYERTHSRIMILSQGLQTLLPLMAFWWLLASLANLALPPTINLLGELSVLVTTFSWSNITLLLTGLNMLVTALYSLYMFTTTQWGSLTHHINNMKPSFTRENTLMFMHLSPILLLSLNPDIITGFSS

SEQ ID NO: 70 - DNA, 3810 bp, Homo sapiens
ccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 71 - protein, 213 aa, Artificial Sequence
  GDNF Protein-Ala-His
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCIAH

SEQ ID NO: 72 - protein, 213 aa, Artificial Sequence
  GDNF Ala-His-Protein
AHMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 73 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 74 - DNA, 3655 bp, Homo sapiens
catacaggccaaaagtctccaagtccctgctaacttcttgctctcgcaacagaatacctatttaggtgggaagaatgaggtgtgggcggcaggctgggtgccgccgccggacgggactttaagatgaagttatgggatgtcgtggctgtctgcctggtgctgctccacaccgcgtccgccttcccgctgcccgccgcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 75 - protein, 187 aa, Artificial Sequence
  GDNF Protein-Ala-His
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCIAH

SEQ ID NO: 76 - protein, 187 aa, Artificial Sequence
  GDNF Ala-His-Protein
AHMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 77 - protein, 185 aa, Homo sapiens
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 78 - DNA, 3830 bp, Homo sapiens
ccaaagcgtccgagactgggtacagtcgtccaggcgtgacgggggcgcggggagccagtgactcctctgggaggggaagggattagggccagaatctctcaaaggtgcaaaaatccagtcaagagagggttttcgggtataccacggaggattaaaactttcaagacaaatgcagtctttgcctaacagcaatggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 79 - protein, 230 aa, Artificial Sequence
  GDNF Protein-Ala-His
MQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCIAH

SEQ ID NO: 80 - protein, 230 aa, Artificial Sequence
  GDNF
AHMQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 81 - protein, 228 aa, Homo sapiens
MQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 82 - DNA, 3751 bp, Homo sapiens
caaagcgtccgagactgggtacagtcgtccaggcgtgacgggggcgcggggagccagtgactcctctgggaggggaagggattagggccagaatctctcaaaggtgcaaaaatccagtcaagagagggttttcgggtataccacggaggattaaaactttcaagacaaatgcagtctttgcctaacagcaatggtgccgccgccggacgggactttaagatgaagttatgggatgtcgtggctgtctgcctggtgctgctccacaccgcgtccgccttcccgctgcccgccgcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 83 - protein, 204 aa, Artificial Sequence
  GDNF Protein-Ala-His
MQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCIAH

SEQ ID NO: 84 - protein, 204 aa, Artificial Sequence
  GDNF Ala-His-Protein
AHMQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 85 - protein, 202 aa, Homo sapiens
MQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 86 - DNA, 3027 bp, Homo sapiens
cagcgttcaggctgccccttcttggctgggaagggcgctgaagaaacaacgcccaggaccaggactatcccctgctcaagctgtgattccgagacccctgccaccactactgcattcacggggatcccaggctagtgggactcgacatgggtagcccccagggcagctccctacagcttgggccatctgcacttttcccaaggccctaagtctccgcctctgggctcgttaaggtttggggtgggagctgtgctgtgggaagcaacccggactacacttggcaagcatggcgctactgaaagtcaagtttgaccagaagaagcgggtcaagttggcccaagggctctggctcatgaactggttctccgtgttggctggcatcatcatcttcagcctaggactgttcctgaagattgaactccgaaagaggagcgatgtgatgaataattctgagagccattttgtgcccaactcattgatagggatgggggtgctatcctgtgtcttcaactcgctggctgggaagatctgctacgacgccctggacccagccaagtatgccagatggaagccctggctgaagccgtacctggctatctgtgttctcttcaacatcatcctcttccttgtggctctctgctgctttctgcttcggggctcgctggagaacaccctgggccaagggctcaagaacggcatgaagtactaccgggacacagacacccctggcaggtgtttcatgaagaagaccatcgacatgctgcagatcgagttcaaatgctgcggcaacaacggttttcgggactggtttgagattcagtggatcagcaatcgctacctggacttttcctccaaagaagtcaaagatcgaatcaagagcaacgtggatgggcggtacctggtggacggcgtccctttcagctgctgcaatcctagctcgccacggccctgcatccagtatcagatcaccaacaactcagcacactacagttacgaccaccagacggaggagctcaacctgtgggtgcgtggctgcagggctgccctgctgagctactacagcagcctcatgaactccatgggtgtcgtcacgctcctcatttggctcttcgaggtgaccattacaattgggctgcgctacctacagacgtcgctggatggtgtgtccaaccccgaggaatctgagagcgagagccagggctggctgctggagaggagcgtgccggagacctggaaggcctttctggagagtgtgaagaagctgggcaagggcaaccaggtggaagccgagggcgcagacgcaggccaggccccagaggctggctgagggccctggggcccctcccctcccgaacactgagaaatagtgcactccaagaaacgtggatctccccctcatccaactccgaaagtctgaatctcccaaggagggcaccatcttacagagactctccctgacggtggaatttaagtttagggtccctaaaagcatttgacacacagttgttgaatgactgacccaaaatgtgaatgaagctaatgtgaatgtgagtgaagctcccttcaggcccgctgccctaggatatgccctcctggtgactcgggggctgtctcagacgactagcccaggacccatctttctcacacggatttagtcccaccctatggccactggccgtatctgagggctgctccccttttagaatttacctcttatgagctccatgttgcttcactctatccaaagtgtcacttggtgcataagcacagaaatctgaaaaatggccatgttgtcttttttttttttttttaatgccaagattgacaggttggccgtttgcttaatgccagaagttgggggaaagttacgcttttctaagaataatggactcttaaggcattgagggctctaaacaggattctttaatcatggagcaagagaatttcaaggcaggggattttatcccccaccaaaaacacagtgaaaggcctgcttttgtgtcccattcacatgccctcggtcactgagtctggagtgaaccacgggttgaggaagtcaggctgttggcgtgtcccagcaccacaccacccctaaagtgccaggtgatctcctgtggctcatcggtggaagcagtggggtaggctgctgccctgctgtggaagaggagcaacaatcagacatgagtccaccctttggagaccaggcctcagctcttggtgggcccagggacacccacacaggtggccatcacagccccatggacaacactaattgtccacagcaaagggcaaggaatcctctgggagcttcttccgtttcttccccccagatacccatcttgaaaaacactatttctggaatgcttctgcatcaaaggagattctttgagatagcccatcttcctgagctagcaaatacaggagttttcactttctttaggaaagagaagctttcaggggaaggagagaatgattttgctgacttcccaagccctggtgaccagaccaaggcagggcccagcataattcctccagttggatgaacattcaagagagctcgttcctacctggctggagaccgaggccagaaggcaaaaaccagaaagggaacagtccataacttacctctgcttctgaccgatggtgtttgggaataggttactttggactgagtttgggttctctgctgtcctaagaacttcagtgtagagaaaataagacttctggtgctgctggggtatgttctgggcttaattcccccaagcagaagaccagatccaagatgtttggacaccctgtcagacgttggtcccaagtttaattagatttctgaatctcgttgaggccaaggaatgatccatactgaaaaaatgctgagccagccatctttggcaaaggtccctgagctcttgctatctctcaagagtgctgagaaccacggtgaaagtgctgctctaggcccacaagtgtaactatgctgttaacagctgtcaatagataattaaaattcatactgtatgaaaatca

SEQ ID NO: 87 - protein, 348 aa, Artificial Sequence
  Peripherin-2 Protein-Ala-His
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESQGWLLERSVPETWKAFLESVKKLGKGNQVEAEGADAGQAPEAGAH

SEQ ID NO: 88 - protein, 348 aa, Artificial Sequence
  Peripherin-2 Ala-His-Protein
AHMALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESQGWLLERSVPETWKAFLESVKKLGKGNQVEAEGADAGQAPEAG

SEQ ID NO: 89 - protein, 346 aa, Homo sapiens
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESQGWLLERSVPETWKAFLESVKKLGKGNQVEAEGADAGQAPEAG

SEQ ID NO: 90 - DNA, 2608 bp, Homo sapiens
tccttcttcattctgcagttggtgccagaactctggatcctgaactggaagaaaatgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgagcatactccagcaagatatgtttttggtagcaaaactgagaaaatcagcttcaggtctgcaatcaaattctgttcaattttagcctgctatatgtcatggttttaacttgcagatgcgcacaattttgcaatgttttacagaaagcactgagttgagcaagcaattcctttatttaaaaaaaaaagtacgtatttagataatcatacttcctctgtgagacaggccataactgaaaaactcttaaatatttagcaatcaaataggaaatgaatgtggacttactaaatggcttttaattcctattataagagcatattttaggtacctatctgctccaattatatttttaacatttaaaaaccaaagtcctctacacttgatttatattatatgtggctttgctgagtcaaggaagtatcatgcaataaggcttaattactaaatgtcaaaccaaactttttctcaaaccagggactatcatctaagattaattacagtaattattttgcgtatacgtaactgctcaaagattatgaatcttatgaatgttaacctttccgtttattacaagcaagtactattatttctgattttataataagaaaatctgtgtttaatcaactgaggcctctcaaccaaataacatctcagagattaagttatatattaaaagcttatgtaacataaaagcaagtacatatagtagtgactatatttaaaaaaacagcataaaatgcttaaaaatgtaatatttactaaaatcagattatgggataatgttgcaggattatactttattgcatcttttttgtttaattgtatttaagcattgtgcaatcacttgggaaaaatattaaattattaacattgaggtattaatacattttaagccttttgtttttaaatttcttttcttccagagattgtttaaaaataaatattgacaaaaat

SEQ ID NO: 91 - protein, 535 aa, Artificial Sequence
  RPE65 Protein-Ala-His
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKSAH

SEQ ID NO: 92 - protein, 535 aa, Artificial Sequence
  RPE65 Ala-His-Protein
AHMSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 93 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS